(19) United States
(12) Patent Application Publication
(10) Pub. No.: US 2010/0080852 A1
(43) Pub. Date: Apr. 1, 2010

(54) PHAMACEUTICAL COMPOSITION COMPRISING NANOPARTICLES AND CASEIN

(76) Inventor: Ronald Arthur Beyerinck, Bend, OR (US)

Correspondence Address: CHERNOFF, VILHAUER, MCCLUNG & STENZEL, LLP, 601 SW Second Avenue, Suite 1600, PORTLAND, OR, 97204-3157, US

(21) Appl. No.: 12/451,289

(22) PCT Filed: Apr. 21, 2008
(86) PCT No.: PCT/IB2008/001135

§ 371 Date: Nov. 3, 2009

Related U.S. Application Data
(60) Provisional application No. 60/915,694, filed on May 3, 2007.

(51) Int. Cl. A61K 9/51; A61K 38/17

(52) U.S. Cl. 424/501; 514/12

(57) ABSTRACT

A pharmaceutical composition comprises nanoparticles comprising a poorly water-soluble drug and a poorly aqueous soluble non-ionizable polymer, and casein.

### BACKGROUND OF THE INVENTION

The present invention relates to compositions comprising nanoparticles comprising a low-solubility drug and a poorly aqueous soluble non-ionizable polymer, and casein or a pharmaceutically acceptable form thereof.

It is known that poorly water-soluble drugs may be formulated as nanoparticles. Nanoparticles are of interest for a variety of reasons, such as to improve the bioavailability of poorly water-soluble drugs, to provide targeted drug delivery to specific areas of the body, to reduce side effects, or to reduce variability in vivo.

A variety of approaches have been taken to formulate drugs as nanoparticles. One approach is to decrease the size of crystalline drug by grinding or milling the drug in the presence of a surface modifier. See, e.g., U.S. Pat. No. 5,145,684. Another approach to forming nanoparticles is to precipitate the drug in the presence of a film forming material such as a polymer. See, e.g., U.S. Pat. No. 5,118,528.

There remain a number of problems associated with the use of nanoparticles to deliver pharmaceutical compounds to the body. The nanoparticles must be stabilized so that they do not aggregate into larger particles in aqueous suspensions. Often surface modifiers such as surfactants are used to stabilize the nanoparticles, but such materials can have adverse physiological effects when administered in vivo. In addition, without a surface modifier present, the surface of the nanoparticles is unprotected, leading to a decrease in performance and stability. Additionally, when formulated as a dry material, the composition should spontaneously form nanoparticles when the composition is added to an aqueous use environment.

Casein has been used as a protective colloid for xanthophylls and other actives. See U.S. Pat. No. 6,863,914 and published U.S. Patent Application No. 2002/0110599A1. Casein has also been included in a long list of surface stabilizers for crystalline and amorphous cyclosporine nanoparticles. See U.S. Pat. No. 6,656,504. Casein has also been used as a protective coating for particles containing a therapeutic agent and a core comprising calcium phosphate. See published U.S. Patent Application No. 200210054914A1. Casein has also been used as a crosslinked matrix for nanoparticles. See U.S. Pat. No. 4,107,288. However, nanoparticles formed from a poorly water soluble drug and casein alone do not adequately solve the problems described above.

Accordingly, there is still a continuing need for nanoparticles that are stable, in the sense of not forming crystalline drug over time or aggregating into larger particles, and that improve the bioavailability of low-Solubility drugs.

### BRIEF SUMMARY OF THE INVENTION

Id one aspect, a solid pharmaceutical composition comprises: (a) nanoparticles comprising a poorly water-soluble drug and a poorly aqueous soluble non-ionizable polymer, wherein (i) the poorly water soluble drug has a solubility in water of less than 5 mg/mL over the pH range of 6.5 to 7.5; (ii) at least 90 wt % of the drug in the nanoparticles is in a non-crystalline form; (iii) the nanoparticles having an average size of less than 500 nm; and (iv) a mass ratio of the poorly water soluble drug to the poorly aqueous soluble non-ionizable polymer is less than 9:1; and (b) casein or a pharmaceutically acceptable form thereof; wherein a mass ratio of (1) casein to (2) the combined mass of the poorly water soluble drug and poorly aqueous soluble non-ionizable polymer is at least 1:20.

In one embodiment, the casein is present in the nanoparticles. In another embodiment, the solid composition comprises a plurality of nanoparticles in a casein matrix. In still another embodiment, the solid composition comprises nanoparticles in a casein matrix wherein casein is elk, present in the nanoparticles.

In another aspect, a pharmaceutical composition comprises an aqueous suspension, the aqueous suspension comprising: (a) nanoparticles comprising a poorly water soluble drug and a poorly aqueous soluble non-ionizable polymer, wherein (i) the poorly water soluble drug has a solubility in water of less than 5 mg/mL over the pH range of 6.5 to 7.5; (ii) at least 90 wt % of the drug in the nanoparticles is in a non-crystalline form; (iii) the nanoparticles have an average size of less than 500 nm; (iv) the poorly water soluble drug and the poorly aqueous soluble non-ionizable polymer constitute at least 60 wt % of the nanoparticles; and (v) a mass ratio of the poorly water soluble drug to the poorly aqueous soluble non-ionizable polymer is less than 9:1; (b) casein or a pharmaceutically acceptable form thereof; and (c) water.

The compositions of the present invention provide a number of advantages over the prior art. Because the pharmaceutical composition comprises (a) nanoparticles comprising a poorly water soluble drug and a non-ionizable polymer, and (b) casein, the stability of the non-crystalline drug in the nanoparticles and the suspension/resuspension stability of the nanoparticles can be addressed independently, resulting in nanoparticles with improved performance and stability.

First, the non-ionizable polymer used in the nanoparticles helps stabilize the poorly water soluble drug. The non-ionizable polymer is chosen so that a portion of the drug is soluble in the non-ionizable polymer. This helps prevent or reduce the rate of crystallization of the non-crystalline drug in the nanoparticle. It is well known that the non-crystalline form of a low-solubility drug provides a greater aqueous concentration of drug relative to the crystalline form of the drug when administered to an aqueous use environment. However, it is also well known that when the drug is not stabilized in the non-crystalline form, the drug rapidly converts to the crystalline form in the use environment. See, for example, Hancock and Parks (Pharmaceutical Research, Vol. 17, No. 4, 2000). Thus, the non-ionizable polymer is selected to maintain the stability of the non-crystalline drug in the nanoparticle, resulting in an enhanced concentration of free drug when the nanoparticle is administered to an aqueous use environment.

Second, the casein helps promote stability of aqueous suspensions of the nanoparticles, reducing, slowing, or preventing agglomeration of the nanoparticles. The use of casein also improves the re-suspendability of solid compositions containing nanoparticles relative to surfactant-based and non-ionizable polymer-based stabilizers: solid compositions of the invention resuspend nanoparticles when administered to an aqueous solution.

Finally, the nanoparticles of the invention may provide improved toleration relative to conventional nanoparticles that incorporate a substantial amount of a surfactant to stabilize the nanoparticles.

The foregoing and other objectives, features, and advantages of the invention will be more readily understood upon consideration of the following detailed description of the invention.

### BRIEF DESCRIPTION OF THE SEVERAL VIEWS OF THE DRAWINGS

FIG. 1. shows schematically a solid composition of the present invention.

### DETAILED DESCRIPTION OF THE INVENTION

The compositions of the present invention relate to (a) a plurality of nanoparticles, each of the nanoparticles comprising the drug and the poorly aqueous soluble non-ionizable polymer, and (b) casein. Pharmaceutical compositions, nanoparticles, non-ionizable polymers, casein, drugs, optional surface stabilizers, and methods for making nanoparticles and the compositions are described in detail below.

### Solid Pharmaceutical Compositions

In one aspect, the invention comprises a solid pharmaceutical composition comprising (a) a plurality of nanoparticles comprising a poorly water-soluble drug and a poorly aqueous soluble non-ionizable polymer, and (b) casein or a pharmaceutically acceptable form thereof. As used herein, the term “solid pharmaceutical composition” means that the composition is in a solid form and substantially free of liquids. Exemplary forms for the solid pharmaceutical composition include particles, granules, powders, dust, pellets, flakes, slabs, rods, and tablets. Methods for making such solid compositions are described herein below.

By “nanoparticles” is meant a plurality of small particles in which the average size of the particles in suspension is less than about 500 nm. In Suspension, by “average size” is meant the effective cumulant diameter as measured by dynamic light scattering, using for example, Brookhaven Instruments' 90Plus particle sizing instrument. By “size” is meant the diameter for spherical particles, or the maximum diameter for non-spherical particles. Preferably, the average size of the nanoparticles is less than 400 nm, more preferably less 300 nm, more preferably lest than 200 nm, and most preferably less than 100 nm.

The width of the particle size distribution in suspension is given by the “polydispersity” of the particles, which is defined as the relative variance in the correlation decay rate distribution, as is known by one skilled in the art. See B. J. Fisken, “Revisiting the method of cumulants for the analysis of dynamic light-scattering data,” Applied Optics, 40(24), 4087-4091 (2001) for a discussion of cumulant diameter and polydispersity. Preferably, the polydispersity of the nanoparticles is less than 0.5. More preferably, the polydispersity of the nanoparticles is less than about 0.3. In one embodiment, the average size of the nanoparticles is lets than 500 nm with a polydispersity of 0.5 or less. In another embodiment, the average size of the nanoparticles is less than 300 nm with a polydispersity of 0.5 or less. In still another embodiment, the average size of the nanoparticles is less than 200 nm with a polydispersity of 0.5 or less. In yet another embodiment, the average size of the nanoparticles is less than 200 nm with a polydispersity of 0.3 or less.

In one embodiment, the casein is present in the nanoparticles together with the poorly water-soluble drug and the non-ionizable polymer. In this embodiment, the casein may act as a surface stabilizer, stabilizing the nanoparticles during the formation process or when present in aqueous suspension, reducing or preventing aggregation or flocculation of the nanoparticles.

In another embodiment, the solid compositions comprise a plurality of nanoparticles in a casein matrix. By “casein matrix” is meant that at least a portion of the nanoparticles in the solid composition are encapsulated by the casein. By “at least a portion of the nanoparticles are encapsulated by the casein” means that the casein encapsulates at least a portion of the plurality of nanoparticles in the composition. The casein may encapsulate only a portion of nanoparticles, or may encapsulate essentially all of the nanoparticles in the composition.

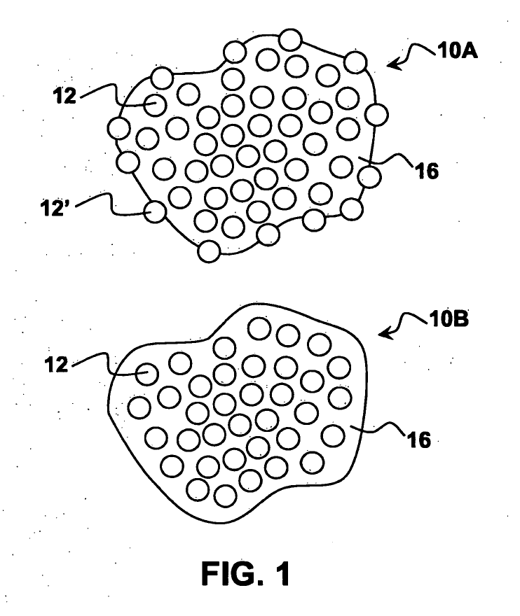

For example, FIG. 1 shows schematically a composition 10A comprising nanoparticles 12 encapsulated by the casein 16. Those nanoparticles 12′ not encapsulated by the casein 16 have at least a portion of their surfaces in contact with the casein 16. Composition 10B has essentially all of the nanoparticles 12 encapsulated with the casein 16.

Thus, the compositions may contain a plurality of nanoparticles, at least a portion of which are encapsulated by the casein; those nanoparticles not encapsulated by the casein are in direct contact with the casein. For compositions comprising nanoparticles in a casein matrix, the presence of nanoparticles in the solid composition can be determined using the following procedure. A sample of the solid composition is embedded in a suitable material, such as an epoxy or polyacrylic acid (e.g., LR White from London Resin Co., London, England). The sample is then microtomed to obtain a cross-section of the solid composition that is about 100 to 200 nm thick. This Sample is then analyzed using transmission electron microscopy (TEM) with energy dispersive X-ray (EDX) analysis. TEM-EDX analysis quantitatively measures the concentration and type of atoms larger than boron over the surface of the sample. From this analysis, regions that are rich in drug and non-ionizable polymer can be distinguished from regions that are rich in casein. The size of the regions that are rich in drug and polymer will have an average diameter of less than 500 nm in this analysis, demonstrating that the solid composition comprises nanoparticles of drug and non-ionizable polymer, and casein. See, for example, Transmission Electron Microscopy and Diffractometry of Materials (2001) for further details of the TEM-EDX method.

Another procedure that demonstrates the solid composition contains nanoparticles is to administer a sample of the solid composition to water to form a suspension of the nanoparticles. The suspension is then analyzed by dynamic light scattering (DLS) as described herein below. A solid composition of the invention will form nanoparticles having an average cumulant diameter of less than 500 nm.

A specific procedure for demonstrating the solid composition contains nanoparticles is as follows. A sample of the solid composition is added to water at ambient temperature such that the concentration of solids is less than about 1 mg/mL. The so-formed suspension is then analyzed by DLS. The solid composition contains nanoparticles if the DLS analysis results in particles having an average cumulant diameter of less than 500 nm.

A solid composition of the invention will show the presence of nanoparticles in at least one, and preferably both of the above tests.

Generally, it is preferred that the solid compositions of the present invention be in the form of small particles or a powder. The small particles or powder may be formed in the process of making the solid composition, or may be formed subsequent to formation of the solid composition. Processes for preparing the compositions of the present invention are discussed herein below.

Preferably, the mean diameter of the small particles of the composition of the present invention will range from about 1 μm to about 500 μm. For improved processing of the solid composition, larger particles are generally preferred. Thus, the mean diameter of the particles is preferably at least 5 μm, more preferably at least 10 μm, or even more preferably at least 25 μm. However, if the particles are too large, the rate of dissolution of the particles can be affected. Thus, the mean diameter may be less than 500 μm, or less than 100 μm in diameter. The mean diameter of the particles preferably ranges from 10 μm to 500 μm, more preferably from 25 μm to 100 μM.

The nanoparticles and casein are collectively present in the solid composition in an amount ranging from about 60 wt % to 100 wt % of the total mass of the composition. Preferably, the nanoparticles and the casein collectively constitute at least 70 wt %, more preferably at least 80 wt %, and even more preferably at least 90 wt % of the composition. In one embodiment, the composition consists essentially of the nanoparticles and the casein. By “consists essentially of” is meant that the composition contains less than 1 wt % of any other excipients and that any such excipients have no affect on the performance or properties of the composition.

The mass ratio of the casein to the mass of the nanoparticles in the composition may range from 1:20 to about 9:1. The casein is preferably present in a sufficient amount so that the nanoparticles re-suspend when the solid composition is administered to an aqueous use environment. Furthermore, preferably a sufficient amount of casein is present to prevent or retard agglomeration of the nanoparticles into larger particles following administration to an aqueous use environment. Thus, the mass ratio of the casein to nanoparticles is at least about 1:20, more preferably at least about 1:15, more preferably at least about 1:10, more preferably at least about 1:7, more preferably at least about 1:5, and most preferably at least about 1:4.

In a preferred embodiment, the solid composition of the present invention has the following composition relative to the total mass of drug, poorly aqueous soluble non-ionizable polymer; and casein in the composition:

1 to 60 wt % drug;

10 to 80 wt % poorly aqueous non-ionizable polymer; and

5 to 50 wt % casein.

In another embodiment, the invention comprises an aqueous suspension comprising a plurality of nanoparticles, casein, and water. Preferably, the casein is associated with the nanoparticles in the suspension. By “associated with” is meant that a portion of the casein in the suspension is in contact with or is adsorbed to the surface portion of the nanoparticles.

Suspensions comprising the nanoparticles, casein, and water may be formed by administering the solid pharmaceutical compositions described above to water or other appropriate aqueous solution. Alternatively, the suspensions may be formed by forming the nanoparticles in an aqueous solution and adding casein. In yet another method, the suspensions may be formed by forming the nanoparticles in an aqueous solution containing casein. These and other methods for forming suspensions of the present invention are described herein below.

### Nanoparticles

The compositions of the present invention comprise a plurality of nanoparticles, each of the nanoparticles comprising the drug and the poorly aqueous soluble non-ionizable polymer. While the drug in its pure form may be either crystalline or non-crystalline, at least 90 wt % of the drug in the nanoparticles is non-crystalline. The term “crystalline,” as used herein, means a particular solid form of a compound that exhibits long-range order in three dimensions. “Non-crystalline” refers to material that does not have long-range three-dimensional order, and is intended to include not only material which has essentially no order, but alto material which may have some small degree of order, but the order is in less than three dimensions and/or is only over short distances. Another term for a non-crystalline form of a material is the “amorphous” form of the material. It has been found that for poorly water-soluble drugs having poor bioavailability that bioavailability improves as the fraction of drug present in the non-crystalline state in the nanoparticle increases. As previously discussed, the non-crystalline form of a low-solubility drug is preferred as it provides a greater aqueous concentration of drug relative to the crystalline form of the drug in an aqueous use environment. Preferably at least about 95 wt % of the drug in the nanoparticle is non-crystalline; in other words, the amount of drug in crystalline form does not exceed about 5 wt %. Amounts of crystalline drug may be measured by Powder X-Ray Diffraction (PXRD), by Differential Scanning Calorimetry (DSC), by solid state nuclear magnetic resonance (NMR), or by any other known quantitative measurement.

The non-crystalline drug in the nanoparticle can exist as a pure phase, as a solid solution of drug homogeneously distributed throughout the non-ionizable polymer, or any combination of these states or those states that lie between them. Preferably, at least a portion of the drug and the non-ionizable polymer is present in the nanoparticle in the form of a solid solution. The solid solution may be thermodynamically stable, in which the drug is present at less than the solubility limit of the drug in the non-ionizable polymer, or may be a supersaturated solid solution in which the drug exceeds its solubility limit in the non-ionizable polymer. Preferably essentially all of the drug and the non-ionizable polymer is present as a solid solution.

The nanoparticles can exist in a number of different configurations. In one embodiment, the nanoparticles comprise a core, the core comprising the non-crystalline drug and the poorly aqueous soluble non-ionizable polymer. As used herein, the term “core” refers to the interior portion of the nanoparticle. The nanoparticles also have a “surface portion,” meaning the outside or exterior portion of the nanoparticle. Thus, the nanoparticles consist of a core (i.e., the interior portion) and a surface portion. In some embodiments, described herein below, materials may be adsorbed to the surface portion of the nanoparticle. Materials adsorbed to the surface portion of the nanoparticle are considered part of the nanoparticle, but are distinguishable from the core of the nanoparticle. Methods to distinguish materials present in the core versus materials adsorbed to the surface portion of the nanoparticle include (1) thermal methods, such as differential scanning calorimetry (DSC); (2) spectroscopic methods, such as X-ray photoelectron spectroscopy (XPS), transmission electron microscopy (TEM) with energy dispersive X-ray (EDX) analysis, fourier transform infra red (FTIR) analysis, and raman Spectroscopy; (3) chromatographic techniques, such as high performance liquid chromatography (HPLC), and gel-permeation chromatography (GPC); and (4) other techniques known in the art.

In one embodiment, the non-crystalline drug and the poorly aqueous soluble non-ionizable polymer constitute at least 60 wt % of the core, more preferably at least 80 wt % of the core. In another embodiment, the core consists essentially of the non-Crystalline drug and the poorly aqueous soluble non-ionizable polymer.

The non-crystalline drug present in the core can exist in non-crystalline pure drug domains, as a thermodynamically stable solid solution of non-crystalline drug homogeneously distributed throughout the non-ionizable polymer, as a supersaturated solid solution of non-crystalline drug homogeneously distributed throughout the non-ionizable polymer, or any combination of these states or those states that lie between them. When the glass-transition temperature (T_{g}) of the non-crystalline drug is different from the T_{g} of the pure polymer by at least about 20° C., the core may exhibit a T_{g} that is different from the T_{g} of pure non-crystalline drug or pure polymer. Preferably, less than 20 wt % of the drug is present in non-crystalline drug domains, with the remaining drug homogeneously distributed throughout the non-ionizable polymer.

In yet another embodiment, the core comprises the non-crystalline drug, the poorly aqueous soluble non-ionizable polymer, and casein or a pharmaceutically acceptable form thereof. The core may be (1) a homogeneous molecular mixture of drug, non-ionizable polymer, and casein, (2) domains of pure drug, domains of pure non-ionizable polymer, and domains of pure casein distributed throughout the core, or (3) any combination of these states or those states that lie between them. In one embodiment, the drug, non-ionizable polymer, and casein are homogeneously distributed throughout the core as a supersaturated solid solution. In another embodiment, the surface portion of the nanoparticle has a higher concentration of casein relative to the nanoparticle as a whole.

In still another embodiment, the core comprises the non-crystalline drug and the poorly aqueous soluble non-ionizable polymer, with the casein adsorbed to the surface portion of the nanoparticle.

In yet another embodiment, the core comprises the non-crystalline drug, the poorly aqueous soluble non-ionizable polymer, and a portion of the casein. The remaining portion of the casein is adsorbed to the surface portion of the nanoparticle. In this embodiment, a portion of the casein is integral to the core, while the remaining portion of casein is adsorbed to the surface portion of the nanoparticle.

The mass ratio of drug to non-ionizable polymer in the nanoparticle can range from about 1:999 to about 9:1 (that is, from about 0.1 wt % drug to 90 wt % drug relative to the total mass of drug and non-ionizable polymer in the nanoparticle). Preferably, the mass ratio of drug to non-ionizable polymer ranges from about 1:99 to about 4:1 (that is, from about 1 wt % to about 80 wt % drug relative to the total mass of drug and non-ionizable polymer), more preferably from about 1:19 to about 3:1 (that is, from about 5 wt % to about 75 wt %), even more preferably from about 1:9 to about 2:1 (that is, from about 10 wt % to about 67 wt % drug relative to the total mass of drug and non-ionizable polymer in the nanoparticle), and most preferably from about 1:3 to about 3:2 (that is, from about 25 wt % to about 60 wt % drug relative to the total mass of drug and non-ionizable polymer in the nanoparticle). In one embodiment, the mass ratio of drug to non-ionizable polymer is lest than 9:1, preferably less than 4:1, more preferably less than 3:1, and most preferably less than 3:2. In another embodiment, the mass ratio of drug to non-ionizable polymer is at least 1:999, preferably at least 1:99, more preferably at least 1:9, and most preferably at least 1:3.

To minimize the total mass of the formulation, high drug loadings are desired. However, if the amount of drug in the nanoparticle is too high, the nanoparticle suspension becomes unstable, resulting in crystallization of the drug in the suspension. Additionally, high amounts of drug in the nanoparticle can lead to crystalline drug formation when the nanoparticles are isolated from suspension in solid form. In absolute terms, it is generally preferred that the amount of drug in the nanoparticle be less than about 90 wt %, more preferably lets than about 80 wt %, even more preferably less than about 75 wt % the total mass of the nanoparticle.

### Non-Ionizable Polymers

The term “polymer” is used conventionally, meaning a compound that is made of monomers connected together to form a larger molecule. A polymer generally consists of at least about 20 monomers connected together. Thus, the molecular weight of the polymer generally will be about 2000 daltons or more. The polymer should be inert, in the sense that it does not chemically react with the drug in an adverse manner, and should be pharmaceutically acceptable.

The polymer is non-ionizable, meaning that the polymer possesses substantially no ionizable functional groups. By “substantially no ionizable functional groups” is meant that the number of ionizable groups covalently attached to the polymer is less than about 0.05 milliequivalents per gram of polymer. Preferably, the number is less than about 0.02 milliequivalents per gram of non-ionizable polymer. By “ionizable groups” is meant functional groups that are at least about 10% ionized over at least a portion of the physiologically relevant pH range of 1 to 8. Such groups have pKₐ values of about 0 to 9.

The non-ionizable polymer is poorly aqueous soluble. By “poorly aqueous soluble” is meant that the non-ionizable polymer has a solubility of less than 0.1 mg/mL when administered alone at a concentration of 0.2 mg/mL to a phosphate buffered saline solution (PBS) at pH 6.5. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. A test to determine the aqueous solubility of a non-ionizable polymer may be performed as follows. The non-ionizable polymer is initially present in bulk powder form with average particle sizes of greater than about 1 micron. The non-ionizable polymer alone is administered at a concentration of 0.2 mg/ml to the pH 6.5 PBS and stirred for approximately 1 hour at room temperature. Next, a nylon 0.45 μm filter is weighed, and the non-ionizable polymer solution is filtered. The filter is dried overnight at 40° C., and weighed the following morning. The amount of non-ionizable polymer dissolved is calculated from the amount of non-ionizable polymer added to the pH 6.5 PBS minus the amount of non-ionizable polymer remaining on the filter (mg). The non-ionizable polymer is considered to be poorly aqueous soluble if it has a solubility of less than 0.1 mg/mL in this test. Preferably, when administered at a concentration of 0.2 mg/mL to the pH 6.5 PBS, a poorly aqueous soluble non-ionizable polymer has a solubility of less than 0.07 mg/mL, more preferably less than 0.05 mg/mL, and most preferably less than 0.01 mg/mL.

It is preferred that the non-ionizable polymer be soluble in an organic solvent. Preferably the non-ionizable polymer has a solubility in an organic solvent of at least about 0.1 mg/mL, and preferably at least 1 mg/mL. Preferably the non-ionizable polymer is not crosslinked.

Suitable non-ionizable polymers include substituted cellulosics, and non-cellulosics. By “cellulosic” is meant a cellulose polymer that has been modified by reaction of at least a portion of the hydroxyl groups on the cellulose repeating units with a compound to form an ester or an ether substituent.

In order to be poorly aqueous soluble, the non-ionizable polymer must be hydrophobic, meaning that the polymer has a sufficient number of hydrophobic groups relative to hydrophilic groups. In a preferred embodiment, the poorly aqueous soluble non-ionizable cellulosic polymer has an ether- or ester-linked alkyl substituent. Suitable alkyl substituents include C₁ to C₄ alkyl groups. Exemplary ether-linked substituents include methyl, ethyl, propyl, and butyl groups. Exemplary ester-linked substituents include acetate, propionate, and butyrate groups.

Exemplary poorly aqueous soluble nonionizable substituted cellulosics include ethylcellulose, propylcellulose, butylcellulose, cellulose acetate, cellulose propionate, cellulose butyrate, cellulose acetate propionate, cellulose acetate butyrate, methyl cellulose acetate, methyl cellulose propionate, methyl cellulose butyrate, ethyl cellulose acetate, ethyl cellulose propionate, ethyl cellulose butyrate, low-substituted hydroxypropyl cellulose, hydroxypropyl methylcellulose acetate, hydroxypropyl methylcellulose propionate, and hydroxypropyl methylcellulose butyrate. Preferably the poorly aqueous soluble non-ionizable polymer is selected from the group consisting of ethyl cellulose, cellulose acetate, and cellulose acetate butyrate.

Exemplary poorly aqueous soluble non-ionizable non-cellulosic polymers include vinyl polymers and co polymers, such as poly(vinyl acetate), poly(vinyl acetate-co-vinyl alcohol), and poly(ethylene-co-vinyl acetate); polymethacrylate and polyacrylate polymers and copolymers, such as polyethyl acrylate-methyl methacrylate) (2:1 monomer ratio), available as EUDRAGIT® NE; polylactones, such as poly(lactide), poly(glycolide), poly(ε-caprolactone), and copolymers of these, including poly(lactide-co-glycolide), poly(lactide-co-ε-caprolactone), poly(ethylene oxide-co-ε-caprolactone), poly(ethylene oxide-co-lactide), and poly(ethylene oxide-co-lactide-co-glycolide); and poly(alkyl)cyanoacrylates, such as poly(isobutyl)cyanoacrylate, and poly(hexyl)cyanoacrylate.

In one embodiment, the non-ionizable polymer is selected from the group consisting of ethylcellulose, propylcellulose, butylcellulose, cellulose acetate, cellulose propionate, cellulose butyrate, cellulose acetate propionate, cellulose acetate butyrate, methyl cellulose acetate, methyl cellulose propionate, methyl cellulose butyrate, ethyl cellulose acetate, ethyl cellulose propionate, ethyl cellulose butyrate, low-substituted hydroxypropyl cellulose, hydroxypropyl methylcellulose acetate, hydroxypropyl methylcellulose propionate, hydroxypropyl methylcellulose butyrate, poly(vinyl acetate), poly(vinyl acetate-co-vinyl alcohol); poly(ethylene-co-vinyl acetate), poly(ethyl acrylate-methyl methacrylate), poly(lactide), poly(glycolide), poly(ε-caprolactone), poly(lactide-co-glycolide), poly(lactide-co-ε-caprolactone), poly(ethylene oxide-co-ε-caprolactone), poly(ethylene oxide-co-lactide), poly(ethylene oxide-co-lactide-co-glycolide), poly(isobutyl)cyanoacrylate, and poly(hexyl)cyanoacrylate.

In another embodiment, the non-ionizable polymer is selected from the group consisting of ethylcellulose, cellulose acetate, cellulose propionate, cellulose butyrate, cellulose acetate butyrate, and poly(ethylene-oxide-co-ε-caprolactone).

In still another embodiment, the non-ionizable polymer is selected from the group consisting of ethylcellulose and poly(ethylene-oxide-co-ε-caprolactone). In another embodiment, the non-ionizable polymer is ethylcellulose. In another embodiment, the non-ionizable polymer is poly(ethylene-oxide-co-ε-caprolactone).

### Surface Stabilizers

The nanoparticles of the present invention may optionally comprise a surface stabilizer in addition to the drug and the non-ionizable polymer. The purpose of the surface stabilizer is to reduce or prevent aggregation or flocculation of the nanoparticles in an aqueous suspension, resulting in nanoparticles with improved stability. In one embodiment, the surface stabilizer is used to stabilize the nanoparticles during the formation process. The stabilizer should be inert, in the sense that it does not chemically react with the drug in an adverse manner, and should be pharmaceutically acceptable.

When a surface stabilizer is present, it may constitute from 0.1 wt % to about 40 wt % of the total mass of the nanoparticles. Generally, lower concentrations of surface stabilizer are preferred. Thus, preferably the surface stabilizer constitutes about 35 wt % or less, more preferably about 30 wt % or less, and most preferably about 25 wt % or less the total mass of the nanoparticles.

In one embodiment, the poorly water soluble drug, the non-ionizable polymer, the optional surface stabilizer, and the casein constitute at least 90 wt % of the solid composition of the invention. In another embodiment, the solid composition of the invention consists essentially of the poorly water soluble drug, the non-ionizable polymer, the optional surface stabilizer, and the casein.

In one embodiment, the surface stabilizer is an amphiphilic compound, meaning that it has both hydrophobic and hydrophilic regions. In another embodiment, the surface stabilizer is a surfactant, including anionic, cationic, zwitterionic, and non-ionic surfactants. Mixtures of surface stabilizers may also be used.

Exemplary surface stabilizers include casein, caseinates, polyvinyl pyrrolidone (PVP), polyoxyethylene alkyl ethers, polyoxyethylene stearates, polyoxyethylene castor oil derivatives, poly(ethylene oxide-propylene oxide) (also known as poloxamers), tragacanth, gelatin, polyethylene glycol, bile salts (such as salts of dihydroxy cholic acids, including sodium and potassium salts of cholic acid, glycocholic acid, and taurocholic acid), phospholipids (such as phosphatidyl cholines, including 1,2-diacylphosphatidylcholine also referred to as PPC or lecithin), sodium dodecylsulfate (also known as sodium lauryl sulfate), benzalkonium chloride, sorbitan esters, polyoxyethylene alkyl ethers, polyoxyethylene castor oil derivatives, polyoxyethylene sorbitan fatty acid esters (polysorbates), polyoxyethylene stearates, triethanolamine, sodium docusate, sodium stearyl fumarate, sodium cyclamate, and mixtures and pharmaceutically acceptable forms thereof.

When casein is used as a surface stabilizer, the casein may be present during the formation of the nanoparticles, or added following formation of the nanoparticles, as discussed herein below. The amount of casein required to stabilize the nanoparticles should generally be at least 5 wt % of the total mass of the nanoparticles, preferably at least 10 wt % of the nanoparticles. When casein is used as a surface stabilizer, additional casein, may be included in the composition such that the nanoparticles are present in a casein matrix, as described herein above.

### Casein

The compositions of the present invention also comprise casein or a pharmaceutically acceptable form thereof. As used herein, the term “casein” refers to phosphoproteins occurring in milk, cheese, and other natural products. The term casein also includes so-called vegetable caseins, also known as legumin or avenin. Vegetable caseins are found in beans and nuts, and are globulin proteins resembling caseins present in milk. Caseins are small proteins with molecular weights ranging from about 10,000 Daltons to about 50,000 Daltons. The casein content of bovine milk represents about 80% of milk proteins, while caseins represent only about 40% of the protein in human milk. Caseins are typically obtained from milk by precipitation at pH 4.6 at 20° C. Under these conditions, the proteins that precipitate are called caseins. There are four main proteins in bovine casein: αₛ₁-casein, αₛ₂-casein, β-casein, and κ-casein.

The caseins are amphiphilic, possessing relatively hydrophobic regions and relatively hydrophilic regions. As a result, caseins are highly surface active. Caseins are sparingly soluble in water, and typically exist in a colloidal particle known as a casein micelle. It is believed that κ-casein is located on the surface of the micelle and contributes to the stability and structure of the micelle. See for example Proteins in Food Processing, (Chapter 3, “The Caseins,” P. F. Fox and A. L. Kelly, Woodhead Publishing Limited, 2004).

As used herein, by “a pharmaceutically acceptable form thereof” is meant either an acid or base addition salt of casein. One preferred form of casein is caseinates. “Caseinates” are produced by reaction of casein with an alkaline substance. Exemplary caseinates include sodium caseinate, calcium caseinate, potassium caseinate and ammonium caseinate.

In one embodiment, the casein is a mixture of caseins found in milk. In another embodiment, the casein is a mixture of caseins found in bovine milk. In still another embodiment, the casein is αₛ₁-casein. In still another embodiment, the casein is αₛ₂-casein. In still another embodiment, the casein is β-casein. In yet another embodiment, the casein is κ-casein. In yet another embodiment, the casein is present as a pharmaceutically acceptable salt form, such as sodium caseinate, calcium caseinate, potassium caseinate or ammonium caseinate. In still another embodiment, the casein is selected from the group consisting of αₛ₁-casein, αₛ₂-casein, β-casein, κ-casein, vegetable casein, sodium caseinate, calcium caseinate, potassium caseinate, ammonium caseinate, and mixtures thereof.

### The Drug

The drug is a “poorly water soluble drug,” meaning that the drug has a solubility in water (over the pH range of 6.5 to 7.5 at 25° C.) of less than 5 mg/mL. The utility of the invention increases as the water solubility of the drug decreases. The drug may have an even lower solubility in water, such as less than about 1 mg/mL, less than about 0.1 mg/mL, and even less than about 0.01 mg/mL.

In general, it may be said that the drug has a dose-to-aqueous solubility ratio greater than about 10 mL, and more typically greater than about 100 mL, where the aqueous solubility (mg/mL) is the minimum value observed in any physiologically relevant aqueous solution (i.e., solutions with pH 1-8), including USP simulated gastric and intestinal buffers, and dose is in mg. Thus, a dose-to-aqueous solubility ratio may be calculated by dividing the dose (in mg) by the aqueous solubility (in mg/mL).

Preferred classes of drugs include, but are not limited to, compounds for use in the following therapeutic areas: antihypertensives, antianxiety agents, antiarrythmia agents, anticlotting agents, anticonvulsants, blood glucose-lowering agents, decongestants, antihistamines, antitussives, antineoplastics, beta blockers, anti-inflammatories, antipsychotic agents, cognitive enhancers, anti-atherosclerotic agents, cholesterol-reducing agents, triglyceride-reducing agents, antiobesity agents, autoimmune disorder agents, anti-impotence agents, antibacterial and antifungal agents, hypnotic agents, anti-Parkinsonism agents, anti-Alzheimer's disease agents, antibiotics, anti-angiogenesis agents, anti-glaucoma agents, anti-depressants, and antiviral agents.

Each named drug should be understood to include the neutral form of the drug or pharmaceutically acceptable forms of the drug. By “pharmaceutically acceptable forms” is meant any pharmaceutically acceptable derivative or variation, including stereoisomers, stereoisomer mixtures, enantiomers, solvates, hydrates, isomorphs, polymorphs, pseudomorphs, neutral forms, salt forms and prodrugs.

Exemplary drugs suitable for use in the nanoparticles include, but are not limited to, phosphodiesterase inhibitors, such as sildenafil and sildenafil citrate; HMG-CoA reductase inhibitors, such as atorvastatin, lovastatin, simvastatin, pravastatin, fluvastatin, rosuvastatin, itavastatin, nisvastatin, visastatin, atavastatin, bervastatin, compactin, dihydrocompactin, dalvastatin, fluindostatin, pitivastatin, and velostatin (also referred to as synvinolin); vasodilator agents, such amiodarone; antipsychotics, such as ziprasidone; calcium channel blockers, such as nifedipine, nicardipine, verapamil, and amlodipine; cholesteryl ester transfer protein (CETP) inhibitors; cyclooxygenase-2 inhibitors; microsomal triglyceride transfer protein (MTP) inhibitors; vascular endothelial growth factor (VEGF) receptor inhibitors; carbonic anhydrase inhibitors; and glycogen phosphorylase inhibitors. Other low-solubility drugs suitable for use in the nanoparticles are disclosed in US Published patent application 2005/0031692, herein incorporated by reference.

In one embodiment, the drug is ziprasidone or a pharmaceutically acceptable form thereof.

In another embodiment, the drug is a hydrophobic non-ionizable drug. By “hydrophobic non-ionizable drug” is meant a subclass of non-ionizable drugs that are essentially water insoluble and highly hydrophobic, and are characterized by a set of physical properties, as described hereinafter. By “non-ionizable” is meant that the drug has substantially no ionizable groups. By “ionizable groups” is meant functional groups that are at least about 10% ionized over at least a portion of the physiologically relevant pH range of 1 to 8. Such groups have pKa values of about 0 to 9. Thus, hydrophobic non-ionizable drugs do not have a pKa value between 0 and 9.

The first property of hydrophobic drugs is that they are extremely hydrophobic. Log P, defined as the base 10 logarithm of the ratio of the drug solubility in octanol to the drug solubility in water, is a widely accepted measure of hydrophobicity. By “extremely hydrophobic” is meant that the Log P value of the drug is at least 4.0, preferably at least 4.5, and most preferably at least 5.0. Log P may be measured experimentally or calculated using methods known in the art. When using a calculated value for Log P, the highest value calculated using any generally accepted method for calculating Log P is used. Calculated Log P values are often referred to by the calculation method, such as Clog P, Alog P, and Mlog P. The Log P may also be estimated using fragmentation methods, such as Crippen's fragmentation method (27 J. Chem. Inf. Comput. Sci. 21 (1987)); Viswanadhan's fragmentation method (29 J. Chem. Inf. Comput. Sci. 163 (1989)); or Broto's fragmentation method (19 Eur. J. Med. Chem.-Chim. Theor. 71 (1984). Preferably the Log P value is calculated by using the average value estimated using Crippen's, Viswanadhan's, and Broto's fragmentation methods.

The second property of hydrophobic drugs is that they have an extremely low solubility in water over the pH range of 6.5 to 7.5 at 25° C. By “extremely low solubility in water” is meant that the solubility of the drug in water is less than 100 μg/mL. Preferably, the hydrophobic drug has a water solubility of less than 50 μg/mL, and most preferably less than 10 μg/mL.

In another embodiment the drug is a cholesteryl ester transfer protein (CETP) inhibitor. CETP inhibitors are drugs that inhibit CETP activity. The effect of a drug on the activity of CETP can be determined by measuring the relative transfer ratio of radiolabeled lipids between lipoprotein fractions, essentially as previously described by Morton in J. Biol. Chem. 256, 11992, 1981 and by Dias in Clin. Chem. 34, 2322, 1988, and as presented in U.S. Pat. No. 6,197,786, the disclosures of which are herein incorporated by reference. The potency of CETP inhibitors may be determined by performing the above-described assay in the presence of varying concentrations of the test compounds and determining the concentration required for 50% inhibition of transfer of radiolabeled lipids between lipoprotein fractions. This value is defined as the “IC₅₀ value.” Preferably, the CETP inhibitor has an IC₅₀ value of less than about 2000 nM, more preferably less than about 1500 nM, even more preferably less than about 1000 nM, and most preferably less than about 500 nM.

Specific examples of CETP inhibitors include [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester; (2R)-3-[[3-(4-chloro-3-ethylphenoxy)phenyl][[3-(1,1,2,2-tetrafluoroethoxy)phenyl]methyl]amino]-1,1,1-trifluoro-2-propanol; S-[2-([[1-(2-ethylbutyl)cyclohexyl]carbonyl]amino)phenyl p-methylpropanethioate; trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid; trans-(4-{[N-(2-{[N′-[3,5-bis(trifluoromethyl)benzyl]-N′-(2-methyl-2H-tetrazol-5-yl)amino]methyl}-5-methyl-4-trifluoromethylphenyl)-N-ethylamino]methyl}cyclohexypacetic acid methanesulfonate; trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide; methyl N-(3-cyano-5-trifluoromethylbenzyl)-[6-(N′-cyclopentylmethyl-N′-ethylamino)indan-5-ylmethyl]-carbamate; methyl (3-cyano-5-trifluoromethylbenzyl)-[6-(N-cyclopentylmethyl-N-ethylamino)indan-5-ylmethyl]-carbamate; ethyl 4-((3,5-bis(trifluoromethyl)phenyl)(2-methyl-2H-tetrazol-5-yl)methyl)-2-ethyl-6-(trifluoromethyl)-3,4-dihydroquinoxaline-1(2H)-carboxylate; tert-butyl 5-(N-(3,5-bis(trifluoromethyl)benzypacetamido)-7-methyl-8-(trifluoromethyl)-2,3,4,5-tetrahydrobenzo[b]azepine-1-carboxylate; (3,5-bis-trifluoromethyl-benzyl)-[2-(cyclohexyl-methoxy-methyl)-5-trifluoromethyl-benzyl]-(2-methyl-2H-tetrazol-5-yl)-amine; 1-[1-(2-{[(3,5-bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-bis-trifluoromethyl-benzyl)-[2-(1-methoxy-cycloheptyl)-5-trifluoromethyl-benzyl]-(2-methyl-2H-tetrazol-5-yl)-amine; (3,5-bis-trifluoromethyl-benzyl)-[2-(1-cyclohexyl-1-methoxy-ethyl)-5-trifluoromethyl-benzyl]-(2-methyl-2H-tetrazol-5-yl)-amine; the drugs disclosed in commonly owned U.S. patent application Ser. Nos. 09/918,127 and 10/066,091, the disclosures of both of which are incorporated herein by reference; and the drugs disclosed in the following patents and published applications, the disclosures of all of which are incorporated herein by reference: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; JP 11049743; WO 0018721; WO 0018723; WO 0018724; WO 0017164; WO 0017165; WO 0017166; EP 992496; EP 987251; WO 9835937; JP 03221376; WO 04020393; WO 05095395; WO 05095409; WO 05100298; WO 05037796; WO 0509805; WO 03028727; WO 04039364; WO 04039453; WO 0633002; and U.S. Provisional Patent Application Nos. 60/781,488 and 60/780,993, both of which were filed on Mar. 10, 2006.

Thus, in one embodiment, the CETP inhibitor is selected from the group of compounds mentioned above. In another embodiment, the CETP inhibitor is selected from the group consisting of (2R)-3-[[3-(4-chloro-3-ethylphenoxy)phenyl][[3-(1,1,2,2-tetrafluoroethoxy)phenyl]methyl]amino]-1,1,1-trifluoro-2-propanol; trans-(2R,4S)-2-(4-{4-[(3,5-bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide amine; (3,5-bis-trifluoromethyl-benzyl)-[2-(cyclohexyl-Methoxy-methyl)-5-trifluoromethyl-benzyl]-(2-methyl-2H-tetrazol-5-yl)-amine; 1-[1-(2-{[(3,5-bis-trifluoromethyl-benzyl)-(2-methyl-2H-tetrazol-5-yl)-amino]-methyl}-4-trifluoromethyl-phenyl)-2-methyl-propyl]-piperidine-4-carboxylic acid; (3,5-bis-trifluoromethyl-benzyl)-[2-(1-methoxy-cycloheptyl)-5-trifluoromethyl-benzyl]-(2-methyl-2H-tetrazol-5-yl)-amine; (3,5-bis-trifluoromethyl-benzyl)-[2-(1-cyclohexyl-1-methoxy-ethyl)-5-trifluoromethyl-benzyl]-(2-methyl-2H-tetrazol-5-yl)-amine; and pharmaceutically acceptable forms thereof.

In still another embodiment, the CETP inhibitor is (2R)-3-[[3-(4-chloro-3-ethylphenoxy)phenyl][[3-(1,1,2,2-tetrafluoroethoxy)phenyl]methyl]amino]-1,1,1-trifluoro-2-propanol.

In still another embodiment, the CETP inhibitor is trans-(2R,4S)-2-(4-{4-[(3,5-Bis-trifluoromethyl-benzyl)-(2-methyl-2H-tetrazol-5-yl)-amino]-2-ethyl-6-trifluoromethyl-3,4-dihydro-2H-quinoline-1-carbonyl}-cyclohexyl)-acetamide.

In another aspect, the drug is an inhibitor of cyclooxygenase-2 (COX-2). COX-2 inhibitors are nonsteroidal anti-inflammatory drugs that exhibit anti-inflammatory, analgesic and antipyretic effects. Preferably, the COX-2 inhibitor is a selective COX-2 inhibitor, meaning that the drug is able to inhibit COX-2 without significant inhibition of cyclooxygenase-1 (COX-1). Preferably, the COX-2 inhibitor has a potency such that the concentration of drug that inhibits 50% of COX-2 enzyme in an in vitro test (i.e., the IC₅₀ value) is less than about 10 μM, preferably less than 5 μM, more preferably less than 2 μM. In addition, it is also preferable that the COX-2 inhibitor be selective relative to COX-1. Thus, preferably, the ratio of the IC_{50.COX-2} to IC_{50.COX-1}, ratio for the compound is less than 0.5, more preferably less than 0.3, and most preferably less than 0.2.

Specific examples of COX-2 inhibitors include 4-(5-(4-methylphenyl)-3-(trifluoromethyl)-1H-pyrazol-1-yl)benzenesulfonamide (celecoxib); 4-(5-methyl-3-phenylisoxazol-4-yl)benzenesulfonamide (valdecoxib); N-(4-(5-methyl-3-phenylisoxazol-4-yl)phenylsulfonyl)propionamide (paracoxb); sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; 2-[(2-chloro-6-fluorophenyl)amino]-5-methyl benzeneacetic acid (lumiracoxib); 4-(3-(difluoromethyl)-5-(3-fluoro-4-methoxyphenyl)-1H-pyrazol-1-yl)benzenesulfonamide (deracoxib); 4-(4-(methylsulfonyl)phenyl)-3-phenylfuran-2(5H)-one (rofecoxib); 5-chloro-2-(6-methylpyridin-3-yl)-3-(4-(methylsulfonyl)phenyl)pyridine (etoricoxib); 2-(3,4-difluorophenyl)-4-(3-hydroxy-3-methylbutoxy)-5-(4-(methylsulfonyl)phenyl)pyridazin-3(2H)-one; (Z)-3-((3-chlorophenyl)(4-(methylsulfonyl)phenyl)methylene)-dihydrofuran-2(3H)-one; N-(2-(cyclohexyloxy)-4-nitrophenyl)methanesulfonamide; 4-Methyl-2-(3,4-dimethylphenyl)-1-(4-sulfamoyl-phenyl)-1H-pyrrole; 6-((5-(4-chlorobenzoyl)-1,4-dimethyl-1H-pyrrol-2-yl)methyl)pyridazin-3(2H)-one; 4-(4-cyclohexyl-2-methyloxazol-5-yl)-2-fluotobenienesulfonamide (tilmacoxib); 2-(4-Ethoxyphenyl)-4-methyl-1-(4-sulfamoylphenyl)-1H-pyrrole; 4-hydroxy-2-methyl-N-(5-methyl-2-thiazolyl)-2H-1,2-benzothiazine-3-carboxamide-1,1-dioxide (meloxicam); 4-(4-chloro-5-(3-fluoro-4-methoxyphenyl)-1H-pyrazol-1-yl)benzenesulfonamide (cimicoxib), and pharmaceutically acceptable forms thereof; and the compounds disclosed in the following patents and published applications, the disclosures of which are incorporated herein by reference: U.S. Pat. No. 5,466,823, U.S. Pat. No. 5,633,272, U.S. Pat. No. 5,932,598, U.S. Pat. No. 6,034,256, U.S. Pat. No. 6,180,651, U.S. Pat. No. 5,908,858, U.S. Pat. No. 5,521,207, U.S. Pat. No. 5,691,374, WO 99/11605, WO 98/03484, and WO 00/24719.

Preferably the COX-2 inhibitor is selected from the group consisting of celecoxib; valdecoxib; paracoxb; sodium (S)-6,8-dichloro-2-(trifluoromethyl)-2H-chromene-3-carboxylate; sodium (S)-7-tert-butyl-6-chloro-2-(trifluoromethyl)-2H-chromene-3-carboxylate; and pharmaceutically acceptable forms thereof. In one embodiment, the COX-2 inhibitor is celecoxib or pharmaceutically acceptable forms thereof.

### Processes for Forming Nanoparticles

The nanoparticles may be formed by any process that results in formation of nanoparticles comprising non-crystalline drug and a non-ionizable polymer. The drug used to form the nanoparticles may be in a crystalline or non-crystalline form; however, at least 90 wt % of the drug in the resulting nanoparticles is in non-crystalline form.

One process for forming nanoparticles is an emulsification process. In this process, the drug and non-ionizable polymer are dissolved in an organic solvent that is immiscible with an aqueous solution in which the drug and non-ionizable polymer are poorly soluble, forming an organic solution. Solvents suitable for forming the solution of dissolved drug and non-ionizable polymers can be any compound or mixture of compounds in which the drug and the non-ionizable polymer are mutually soluble and which is immiscible in the aqueous solution. As used herein, the term “immiscible” means that the organic solvent has a solubility in the aqueous solution of less than about 10 wt %, preferably less than about 5 wt %, and most preferably less than about 3 wt %. Preferably, the organic solvent is also volatile with a boiling point of 150° C. or less. Exemplary organic solvents include methylene chloride, trichloroethylene, trichloro-trifluoroethylene, tetrachloroethane, trichloroethane, dichloroethane, dibromoethane, ethyl acetate, phenol, chloroform, toluene, xylene, ethyl-benzene, benzyl alcohol, creosol, methyl-ethyl ketone, methyl-isobutyl ketone, hexane, heptane, ether, and mixtures thereof. Preferred organic solvents are methylene chloride, ethyl acetate, benzyl alcohol, and mixtures thereof. The aqueous solution preferably is water.

Once the organic solution is formed, it is then mixed with the aqueous solution and homogenized to form an emulsion of fine droplets of the water immiscible organic solvent distributed throughout the aqueous phase. The volume ratio of organic solution to aqueous solution used in the process will generally range from 1:100 (organic solution:aqueous solution) to 2:3 (organic solution:aqueous solution). Preferably, the organic solution:aqueous solution volume ratio ranges from 1:9 to 1:2 (organic solution:aqueous solution). The emulsion is generally formed by a two-step homogenization procedure. The solution of drug, non-ionizable polymer and organic solvent are first mixed with the aqueous solution using a rotor/stator or similar mixer to create a “pre-emulsion”. This mixture is then further processed with a high-pressure homogenizer that subjects the droplets to very high shear, creating a uniform emulsion of very small droplets. A portion of the organic solvent is then removed forming a suspension of the nanoparticles in the aqueous solution. Exemplary processes for removing the organic solvent include evaporation, extraction, diafiltration, pervaporation, vapor permeation, distillation, and filtration. Preferably, the organic solvent is removed to a level that is acceptable according to The International Committee on Harmonization (ICH) guidelines. Preferably, the concentration of organic solvent in the nanoparticle suspension is less than the solubility of the organic solvent in the aqueous solution. Even lower concentrations of organic solvent are preferred. Thus, the concentration of organic solvent in the nanoparticle suspension may be less than about 5 wt %, less than about 3 wt %, less than 1 wt %, and even less than 0.1 wt %.

An alternative process to form the nanoparticles is a precipitation process. In this process, the drug and non-ionizable polymer are first dissolved in an organic solvent that is miscible with an aqueous solution in which the drug and non-ionizable polymer are poorly soluble to form an organic solution. The organic solution is mixed with the aqueous solution causing the nanoparticles to precipitate. Organic solvents suitable for forming the organic solution of dissolved drug and non-ionizable polymers can be any compound or mixture of compounds in which the drug and the non-ionizable polymer are mutually soluble and which is miscible in the aqueous solution. Preferably, the organic solvent is also volatile with a boiling point of 150° C. or less. Exemplary organic solvents include acetone, methanol, ethanol, tetrahydrofuran (THF), and dimethylsulfoxide (DMSO). Mixtures of organic solvents, such as 50% methanol and 50% acetone, can also be used, as can mixtures with water, so long as the non-ionizable polymer and drug are sufficiently soluble to dissolve the drug and non-ionizable polymer. Preferred organic solvents are methanol, acetone, and mixtures thereof.

The aqueous solution may be any compound or mixture of compounds in which the drug and non-ionizable polymers are sufficiently insoluble so as to precipitate to form nanoparticles. The aqueous solution is preferably water.

The organic solution and aqueous solution are combined under conditions that cause solids to precipitate as nanoparticles. The mixing can be by addition of a bolus or stream of organic solution to a stirring container of the aqueous solution. Alternately a stream or jet of organic solution can be mixed with a moving stream of aqueous solution. In either case, the precipitation results in the formation of a suspension of nanoparticles in the aqueous solution.

For the precipitation process, the amount of drug and polymer in the organic solution depends on the solubility of each in the organic solvent and the desired ratios of drug to polymer in the resulting nanoparticles. The solution may comprise from about 0.1 wt % to about 20 wt % dissolved solids. A dissolved solids content of from about 0.5 wt % to 10 wt % is preferred.

The organic solution:aqueous solution volume ratio should be selected such that there is sufficient aqueous solution in the nanoparticle suspension that the nanoparticles solidify and do not rapidly agglomerate. However, too much aqueous solution will result in a very dilute suspension of nanoparticles, which may require further processing for ultimate use. Generally, the organic solution:aqueous solution volume ratio should be at least 1:100, but generally should be less than 1:2 (organic solution:aqueous solution). Preferably, the organic solution:aqueous solution volume ratio ranges from about 1:20 to about 1:3.

Once the nanoparticle suspension is made, a portion of the organic solvent may be removed from the suspension using methods known in the art. Exemplary processes for removing the organic solvent include evaporation, extraction, diafiltration, pervaporation, vapor permeation, distillation, and filtration. Preferably, the solvent is removed to a level that is acceptable according to ICH guidelines. Thus, the concentration of solvent in the nanoparticle suspension may be less than about 10 wt %, less than about 5 wt %, less than about 3 wt %, less than 1 wt %, and even less than 0.1 wt %.

### Formation of Compositions

The compositions of the present invention comprise nanoparticles comprising a drug and non-ionizable polymer, and casein. The casein can be formulated with the nanoparticles either during the process used to form the nanoparticles or after the nanoparticles are formed.

In one embodiment, the casein is formulated with the nanoparticles during the nanoparticle-formation process. In this embodiment, the casein may be considered to be part of the nanoparticles. For the emulsion and precipitation processes described above, the casein can be either added to the organic solution comprising the drug and non-ionizable polymer or added to the aqueous solution. In a preferred embodiment, the casein is added to the aqueous solution. Formulating the casein in the aqueous solution is advantageous as it allows the casein to help reduce or eliminate flocculation or aggregation of the nanoparticles once they are formed.

Thus, in one embodiment, the compositions of the present invention are formed by the process comprising (a) forming an organic solution comprising a poorly water soluble drug and a non-ionizable polymer dissolved in a water-immiscible solvent, (b) forming an aqueous solution comprising casein, (c) mixing the organic solution and the aqueous solution to form an emulsion, and (d) removing the water-immiscible solvent from the emulsion to form an aqueous suspension comprising nanoparticles comprising the poorly water soluble drug and the non-ionizable polymer, and casein.

In another embodiment, the compositions of the present invention are formed by the process comprising (a) forming an organic solution comprising a poorly water soluble drug and a non-ionizable polymer dissolved in a water-miscible solvent, (b) forming an aqueous solution comprising casein, (c) mixing the organic solution and the aqueous solution to form an aqueous suspension comprising nanoparticles comprising the poorly water soluble drug and the non-ionizable polymer, and casein.

In another embodiment, the casein is formulated with the nanoparticles after the nanoparticles have been formed. This has advantages when the process for removing the solvent from the nanoparticle suspension would also remove the casein (e.g., diafiltration). This embodiment is also preferred when processes are used to increase the concentration of nanoparticles in the suspension. Generally, in this embodiment, casein is administered to the suspension containing the nanoparticles. Note that when the nanoparticles are suspended in an aqueous solution, the casein may not completely dissolve in the water. As discussed above, casein often forms micelles when added to water. In such instances, the casein may be present in the form of micelles.

In still another embodiment, a process for forming nanoparticles, comprises: (a) forming an organic solution comprising a poorly water soluble drug and a poorly aqueous soluble non-ionizable polymer dissolved in an organic solvent, wherein (i) the drug has a solubility in water of less than 5 mg/ml over the pH range of 6.5 to 7.5, and (ii) a mass ratio of the poorly water soluble drug to the non-ionizable polymer is less than 9:1; (b) forming an aqueous solution, wherein the drug and the non-ionizable polymer are poorly soluble in the aqueous solution; (c) mixing the organic solution with the aqueous solution to form a first mixture; (d) removing the organic solvent from the first mixture to form a suspension comprising the nanoparticles and the aqueous solution, wherein (i) the nanoparticles have an average size of less than 500 nm, and (ii) at least 90 wt % of the drug in the nanoparticles is non-crystalline; and (e) adding casein to either the aqueous solution of step (b) or to the suspension of step (d), wherein a mass ratio of (1) the casein to (2) the combined mass of the poorly water soluble drug and the non-ionizable poly mer is at least 1:20. In one embodiment, the process comprises the additional step (f) removing liquid from the suspension to form a solid composition comprising the nanoparticles and the casein.

A variety of processes may be used to form solid compositions comprising nanoparticles comprising a poorly water soluble drug and a non-ionizable polymer, and casein. Essentially any process that removes the liquid from the suspension may be used to form a solid composition, provided the process does not affect the properties of the nanoparticles or casein. Exemplary processes include spray drying, spray coating, spray layering, lyophylization, evaporation, vacuum evaporation, and filtration. A preferred process is spray drying, as described in the Examples. One or more processes may be combined to remove the liquid from the nanoparticle/casein suspension and yield a solid composition. For example, a portion of the liquid may be removed by filtration to concentrate the nanoparticles, followed by spray-drying to remove most of the remaining liquids, followed by a further drying step such as tray-drying.

Once the solid composition is formed, it may be desirable to form small particles of the solid composition, as discussed above. Some of the processes described above, such as spray drying, will typically produce small particles of the solid composition. Other processes used to form the solid composition may result in larger particles, sheets, flakes, or other forms of the solid composition. Thus, the particle size of the solid composition may be adjusted using various techniques known in the art, such as through the use of grinders and mills. See, for example, Remington: The Science and Practice of Pharmacy, 20^{th} Edition (2000).

### Resuspendability

In one embodiment, the solid compositions of the present invention result in improved resuspendability of the nanoparticles relative to surfactant-based and polymer-based stabilizers. The term “resuspendability” as used herein means the ability of the solid material, when administered to an aqueous use environment, to form a nanoparticle suspension.

The ability of the solid composition to resuspend nanoparticles when administered to an aqueous solution can be determined using the following procedures. In the first procedure, the average particle size of the re-suspended material is determined as follows. The solid composition is added to an aqueous solution, such as water, PBS, or MFD solution, to form a suspension. A sample of the solid composition is added to water at ambient temperature such that the concentration of solids is less than about 1 mg/mL. The average particle size of the nanoparticles formed during this (re)suspension is then determined by dynamic light scattering (DLS) techniques. A solid composition is said to provide good resuspendability if, upon administration to an aqueous solution, the average particle size as determined by DLS techniques is at least 50% and no more than 200% the average particle size of the nanoparticles prior to recovery of the solid composition. Preferably, the formulation provides an average particle size that is at least 67% and no more than 150% the average particle size prior to recovery of the solid composition. Even more preferably, the formulation provides an average particle size that is at least 75% and no more than 133% the average particle size prior to recovery of the solid composition.

The second procedure is known as a filter potency test. In this test the concentration of drug after passing the suspension of the nanoparticles through a filter is determined. The solid composition is added to an aqueous solution as described above. The concentration of drug in the so-formed suspension is then determined using standard techniques, such as by high-performance liquid chromatography (HPLC). Next, the suspension is filtered through a filter, and the concentration of drug in the filtered sample is determined via standard techniques. A loss in potency after filtering a sample through a filter is an indication that the nanoparticles in the sample are larger than the filter pore size. Exemplary filters that can be used in this test include a 1-μm glass fiber filter, a 0.45-μm syringe filter, and a 0.2-μm syringe filter. One skilled in the art will understand that the pore size of the filter should be selected to ensure the nanoparticles are not retained on the filter. Generally, the pore size of filter and the range of nanoparticle average diameters are given as follows:

| Filter Pore Size (μm) | Suitable Range of Nanoparticle Diameters (nm) |
|---|---|
| 1 | >250 |
| 0.45 | 150 to 300 |
| 0.2 | <200 |

A solid composition is said to provide good resuspendability if the ratio of the concentration of drug in the filtered sample is at least 60% the concentration of drug in the unfiltered sample. Preferably, the concentration of drug in the filtered sample is at least 70% the concentration of drug in the unfiltered sample. Most preferably, the concentration of drug in the filtered sample is at least 80% the concentration of drug in the unfiltered sample.

In an especially preferred embodiment, a composition provides good resuspendability in both of the tests described above.

### Dosage Forms

The compositions of the present invention may be administered using any known dosage form. The nanoparticles may be formulated for administration via oral, topical, subdermal, intranasal, buccal, intrathecal, ocular, intraaural, subcutaneous spaces, intraarticular, vaginal tract, arterial and venous blood vessels, pulmonary tract or intramuscular tissue of an animal, such as a mammal and particularly a human. Oral dosage forms include: powders or granules; tablets; chewable tablets; capsules; unit dose packets, sometimes referred to in the art as “sachets” or “oral powders for constitution” (OPC); syrups; and suspensions. Parenteral dosage forms include reconstitutable powders or suspensions. Topical dosage forms include creams, pastes, suspensions, powders, foams and gels. Ocular dosage forms include suspensions, powders, gels, creams, pastes, solid inserts and implants.

In one embodiment, the compositions of the present invention are capable of improving the concentration of dissolved drug in a use environment relative to a control composition consisting essentially of the drug alone without any non-ionizable polymer or casein. In order to determine concentration enhancement in vitro, the amount of “free” drug, or solvated drug is measured. By “free” drug is meant drug which is in the form of dissolved drug or present in micelles, but which is not in the nanoparticles or any solid particles larger than 500 nm, such as precipitate. A composition of the invention provides concentration enhancement if, when administered to an aqueous use environment, it provides a free drug concentration that is at least 1.25-fold the free drug concentration provided by the control composition. Preferably, the free drug concentration provided by the compositions of the invention are at least about 1.5-fold, more preferably at least about 2-fold, and most preferably at least about 3-fold that provided by the control composition.

Alternatively, the compositions of the present invention, when dosed orally to a human or other animal, provide an AUC in drug concentration in the blood plasma or serum (or relative bioavailability) that is at least 1.25-fold that observed in comparison to the control composition. Preferably, the blood AUC is at least about 2-fold, more preferably at least about 3-fold, even more preferably at least about 4-fold, still more preferably at least about 6-fold, yet more preferably at least about 10-fold, and most preferably at least about 20-fold that of the control composition. The determination of AUCs is a well-known procedure and is described, for example, in Welling, “Pharmacokinetics Processes and Mathematics,” ACS Monograph 185 (1986).

Alternatively, the compositions of the present invention, when dosed orally to a human or other animal, provide a maximum drug concentration in the blood plasma or serum (Cₘₐₓ) that is at least 1.25-fold that observed in comparison to the control composition. Preferably, the Cₘₐₓ is at least about 2-fold, more preferably at least about 3-fold, even more preferably at least about 4-fold, still more preferably at least about 6-fold, yet more preferably at least about 10-fold, and most preferably at least about 20-fold that of the control composition. Thus, compositions that meet the in vitro or in vivo performance criteria, or both, are considered to be within the scope of the invention.

Without further elaboration, it is believed that one of ordinary skill in the art can, using the foregoing description, utilize the present invention to its fullest extent. Therefore, the following specific embodiments are to be construed as merely illustrative and not restrictive of the scope of the invention. Those of ordinary skill in the art will understand that variations of the conditions and processes of the following examples can be used.

### EXAMPLES

### Drugs Used in Examples

The following drugs were used in the examples described below.

Drug 1 was 4-[5-(4-methylphenyl)-3-(trifluoromethyl)-1H-pyrazol-1-yl]benzenesulfonamide, also known as celecoxib, having the structure:

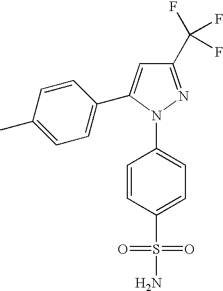

Drug 1 has a solubility in MFD 9 solution of about 40 μg/mL, and a Log P value of 3.75. The Tₘ of Drug 1 is 158° C., and the T_{g} of amorphous Drug 1 was determined by DSC analysis to be 54° C.

Drug 2 was (2R)-3-[[3-(4-chloro-3-ethylphenoxy)phenyl][[3-(1,1,2,2-tetrafluoroethoxy)phenyl]methyl]amino]-1,1,1-trifluoro-2-propanol, having the structure:

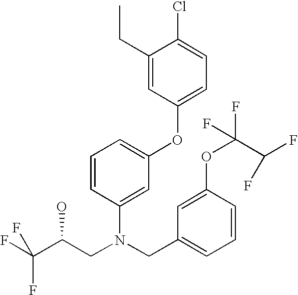

Drug 2 has a solubility in PBS of less than 0.1 μg/mL, and a Log P value of 10. The Tₘ of Drug 2 is 10° C., and the T_{g} was determined by DSC analysis to be −16° C.

Drug 3 was [2R,4S] 4-[acetyl-(3,5-bis-trifluoromethyl-benzyl)-amino]-2-ethyl-6-trifluoromethyl-3,4-dihydro-2H-quinoline-1-carboxylic acid isopropyl ester, having the structure:

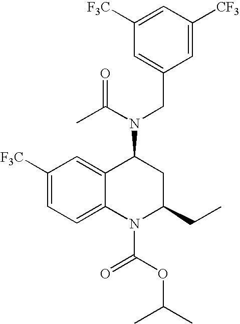

Drug 3 has a solubility in MFD solution of about 11 μg/mL, and a CLog P value of about 6.6. The Tₘ of Drug 3 is 111° C., and the T_{g} was determined by DSC analysis to be about 45° C.

Drug 4 was 2-(2-chloro-4-iodophenylamino)-N-cyclopropylmethoxy-3,4-difluorobenzamide, having the structure:

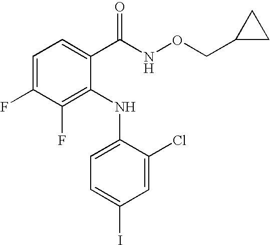

Drug 4 has an aqueous solubility of about 0.03 μg/mL, and a CLog P value of 5.9. The Tₘ of Drug 4 is 177° C., and the T_{g} was determined by DSC analysis to be about 46° C.

Drug 5 was triphenyl bismuth, (C₆H₅)₃Bi (available from Alfa Aesar, Ward Hill, Mass.), having the structure:

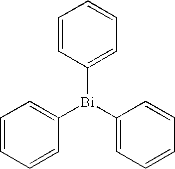

Drug 5 has a Log P value of 6.0. The melting point for Drug 5 is 77.6° C.

### Excipients Used in the Examples

The following poorly aqueous soluble non-ionizable polymers were used in the examples: ethylcellulose (ETHOCEL® Viscosity 4, Dow Chemical Co., Midland, Mich.), and poly(ethylene oxide-co-ε-caprolactone), designated as pCL-PEG (grade P3128-EOCL available from Polymer Source Inc., Montreal, Quebec, Canada), having a polycaprolactone molecular weight of 10,000 and a poly(ethylene oxide) molecular weight of 5000 daltons.

The non-ionizable polymers were evaluated using the following procedure to determine their aqueous solubility. First, 0.2 mg/mL of the non-ionizable polymer was added to a PBS solution consisting of 20 mM Na₂HPO₄, 47 mM KH₂PO₄, 87 mM NaCl, and 0.2 mM KCl, adjusted to pH 6.5 with NaOH. The non-ionizable polymer was stirred in the solution for approximately 1 hour at room temperature. Next, the non-ionizable polymer solution was filtered through a nylon 0.45 μm filter that had been weighed dry prior to filtration. The filter was dried overnight at 40° C., and weighed the following morning. The amount of non-ionizable polymer dissolved was calculated from the amount of non-ionizable polymer added to the PBS minus the amount of non-ionizable polymer remaining on the filter. The results of these tests are shown in Table 1, and show that the non-ionizable polymers are poorly aqueous soluble.

TABLE 1

| Example Non-ionizable | Soluble at pH 6.5 | |
|---|---|---|
| Polymer | (mg/mL) | Observations |
| Ethylcellulose | <0.001 | Fine Particle Suspension |
| pCL-PEG | 0.02 | Fine Particle Suspension |

Sodium caseinate was obtained from several sources: (1) Spectrum Chemicals, Gardena, Calif., (2) American Casein Company, Burlington, N.J., and (3) Sigma Chemicals, St Louis, Mo.

Sodium β-caseinate was formed from β-casein (obtained from Sigma), using the following procedure. First, 400 mg β-casein was added to 80 mL deionized water. Next, 5 mL 0.001 N NaOH, and 12 mL 0.01 N NaOH, were added to reach a pH of 7.02. The solution was lyophilized to obtain solid sodium β-caseinate.

### Example 1

Nanoparticles containing celecoxib (“Drug 1”) were prepared as follows. First, 120 mg celecoxib and 360 mg ethylcellulose were dissolved in 7.5 mL methylene chloride to form an organic solution. Next, 120 mg casein was added to 30 mL deionized water to form an aqueous solution. The organic solution was then poured into the aqueous solution and emulsified for 3 min using a Kinematica Polytron 3100 rotor/stator (Kinematica AG, Lucerne, Switzerland) at 10,000 rpm (high-shear mixing). The solution was further emulsified using a Microfluidizer (Microfluidics [Newton, Mass.] model M-1108 F12Y with ice bath and cooling coil), for 6 minutes (high-pressure homogenization). The methylene chloride was removed from the emulsion using a rotary evaporator, resulting in an aqueous suspension of nanoparticles, with a mass ratio of 20:60:20 celecoxib:ethylcellulose:casein.

### Light Scattering Analysis

The particle size of the nanoparticles in the aqueous suspension was determined using dynamic light scattering (DLS) as follows. First, the aqueous suspension was filtered using a 1 μm glass membrane filter (Anatop filter, Whatman), and poured into a cuvette. Light-scattering was measured using a Brookhaven Instruments (Holtsville, N.Y.) BI-200SM particle size analyzer with a BI-9000AT correlator. The sums of exponentials from the autocorrelation functions are analyzed to extract size distributions from the samples, and the size is reported as the cumulant value. The average diameter was found to be 79 nm, with a polydispersity of 0.16.

The aqueous suspension was allowed to stand unmixed for 2 days (ambient conditions) to measure stability. DLS analysis showed that the average cumulant diameter of the nanoparticle suspension after 2 days was 75 nm, with a polydispersity of 0.14. These results demonstrate that the nanoparticle suspension of Example 1 was stable for at least 2 days with no significant particle agglomeration.

### Isolation of Solid Compositions

A solid composition comprising the nanoparticles and casein was prepared using the following process. First, 150 mg casein (5 mg/mL) was added to the aqueous suspension of Example 1, resulting in a suspension with a mass ratio of 16:48:36 Celecoxib:ethylcellulose:casein.

To form the solid composition of Example 1, the aqueous nanoparticle suspension was added to a reservoir and pumped to a two fluid nozzle located in a spray-drying chamber, using an HPLC pump (model 515, Waters Corp., Milford, Mass.) at a flow rate of about 0.15 g/min. The spray-drying chamber consisted of two sections: a straight-side section (top), and a cone section (bottom). The top of the straight-side section was equipped with a spray-solution inlet. The spray solution was sprayed through the spray-solution inlet using the two-fluid nozzle, into the straight-side section of the spray-drying chamber. The straight-side section had a diameter of 10 cm and a length of 19 cm.

Drying gas (nitrogen) entered the cone section through a drying-gas inlet at a flow of about 1.0 SCFM and an inlet temperature of about 120° C. The flow rate of drying gas and spray solution were selected such that the atomized spray solution was sufficiently dry by the time it reached the walls of the spray-drying chamber that it did not stick to the walls. The diameter of the cone section at the top was 10 cm, and the distance from the top of the cone section to the bottom was 19 cm. At the bottom of the cone section was a 4.7-cm diameter outlet port, fitted with a 0.8 μm nylon filter (Magna, GE Osmonics, Minnetonka, Minn.) supported by a metal screen. The spray dried composition was collected on the filter, and evaporated solvent and drying gas were removed from the spray-drying chamber through the outlet port.

### Nanoparticle Resuspension

The solid composition of Example 1 was resuspended in deionized water as follows. About 40 mg of the solid composition was added to 2 mL of water, vortexed 10 seconds, and sonicated 5 minutes. DLS analysis is summarized in Table 2, and showed that the average cumulant diameter of the nanoparticle suspension was 83 nm, with a polydispersity of 0.14. This demonstrates that the solid composition of Example 1 resulted in the formation of nanoparticles upon resuspension in water.

TABLE 2

| Sample | Diameter (nm) | Polydispersity |
|---|---|---|
| Initial | 79 | 0.16 |
| After Storage | 75 | 0.14 |
| After Resuspension | 83 | 0.14 |

### Example 2

For Example 2, nanoparticles containing celecoxib were prepared as described in Example 1 with the following exceptions. The organic solution consisted of 120 mg celecoxib and 420 mg ethylcellulose dissolved in 6 mL methylene chloride. The aqueous solution consisted of 120 mg sodium β-caseinate (made as described above) in 15 mL deionized water. This process resulted in an aqueous suspension of nanoparticles, with a mass ratio of 18:64:18 celecoxib:ethylcellulose:sodium β-caseinate. DLS analysis showed that the average cumulant diameter of the nanoparticles in suspension was 104 nm, with a polydispersity of 0.19.

### Isolation of Solid Composition

The nanoparticle suspension of Example 2 was spray-dried as described in Example 1 to form a solid composition of Example 2.

### Nanoparticle Resuspension

The solid composition of Example 2 was resuspended by adding a 37.7 mg sample to 4 mL deionized water containing 5 wt % dextrose. DLS analysis showed that the average cumulant diameter of the nanoparticle suspension was 145 nm, with a polydispersity of 0.24. This demonstrates that resuspension of the solid composition of Example 2 resulted in the formation of nanoparticles.

### Control 1

For Control 1, nanoparticles were prepared without casein as follows. First, 120 mg celecoxib and 420 mg ethylcellulose were dissolved in 6 mL methylene chloride to form an organic solution. Next, 120 mg poly-glutamic acid-alanine, sodium salt (poly-Glu-Ala 6:4, mw 30,000, available from Sigma Chemical Co.) was added to 15 mL deionized water to form an aqueous solution. The organic solution was then poured into the aqueous solution and emulsified as described in Example 1. The methylene chloride was removed from the emulsion using a rotary evaporator, resulting in an aqueous suspension of nanoparticles, with a composition ratio of 18:64:18 celecoxib:ethylcellulose:poly-Glu-Ala. DLS analysis showed that the average cumulant diameter of the nanoparticles in suspension was 123 nm, with a polydispersity of 0.12.

The nanoparticles of Control 1 were spray-dried and resuspended as described in Example 2. Following resuspension in deionized water containing 5 wt % dextrose, DLS analysis showed that the average cumulant diameter of the nanoparticles in suspension was 1094 nm, with a polydispersity of 0.58. This large particle size indicates that the solid composition of Control 1 did not result in the formation of nanoparticles.

### Filter Potency

A filter potency test was performed to characterize the resuspended nanoparticles of Example 2 and Control 1. The filter potency test is used to examine changes in nanoparticle suspension potencies due to particle agglomeration. As nanoparticles agglomerate, the larger particles are removed via filtration, and the concentration of drug in the nanoparticle suspension is reduced.

To measure nanoparticle potency, a 100 μL sample of the aqueous nanoparticle suspensions was added to 1 mL 80/20 methanol/acetonitrile, and the concentration of drug in solution was analyzed by high-performance liquid chromatography (HPLC). HPLC analysis of celecoxib was performed using a Zorbax SB C₈ column. The mobile phase consisted of 55% acetonitrile/45% 10 mM ammonium acetate, adjusted to pH 4. UV absorbance was measured at 254 nm.

Samples of the resuspended nanoparticles for Example 2 and Control 1 were filtered through a 1 μm glass membrane filter and diluted in 80/20 methanol/acetonitrile for HPLC analysis. Potencies of the nanoparticle suspensions are shown in Table 3. The results in Table 3 show that while 97% of the potency of the nanoparticle suspension of Example 2 is maintained following filtration by a 1 μm filter, only 10% of the potency of Control 1 remained in suspension. This indicates that most of the nanoparticles of Example 2 remained small and unagglomerated, in contrast to the nanoparticles of the control.

TABLE 3

| | Potency | Potency | |
|---|---|---|---|
| | Unfiltered | 1 μm filtered | Potency |
| Sample | (mg/mL) | (mg/mL) | Retained (%) |
| Example 2 | 1.81 | 1.75 | 97 |
| Control 1 | 2.08 | 0.21 | 10 |

### Examples 3 and 4

The nanoparticles of Examples 3 and 4 were made containing celecoxib, ethylcellulose, and two concentrations of casein. For the nanoparticles of Example 3, 120 mg celecoxib and 360 mg ethylcellulose were dissolved in 7.5 mL methylene chloride to form an organic solution, and 120 mg casein was added to 30 mL deionized water to form an aqueous solution. For the nanoparticles of Example 4, 120 mg celecoxib and 330 mg ethylcellulose were dissolved in 7.5 mL methylene chloride to form an organic solution, and 150 mg casein was added to 30 mL deionized water to form an aqueous solution. The mixtures were emulsified as described in Example 1. The methylene chloride was removed using a rotary evaporator, to obtain the aqueous suspensions of nanoparticles of Examples 3 and 4. The nanoparticles of Example 3 had a mass ratio of 20:60:20 celecoxib:ethylcellulose:casein, and the nanoparticles of Example 4 had a mass ratio of 20:55:25 celecoxib:ethylcellulose:casein. DLS analysis showed that the average cumulant diameter of the nanoparticles of Example 3 was 85 nm, with a polydispersity of 0.10. The average cumulant diameter of the nanoparticles of Example 4 was also 85 nm, with a polydispersity of 0.10.

The aqueous suspensions were allowed to stand unmixed for 24 hours (ambient conditions) to measure stability. DLS analysis showed that the average cumulant diameter of the nanoparticles of Example 3 after 24 hours was 86 nm, with a polydispersity of 0.10. The average cumulant diameter of the nanoparticles of Example 4 after 24 hours was 85 nm, with a polydispersity of 0.10. These results demonstrate that the nanoparticle suspensions of Examples 3 and 4 are stable for at least 24 hours with no measurable particle agglomeration.

### Isolation of Solid Compositions

The nanoparticles of Examples 3 and 4 were spray-dried as described in Example 1.

### Nanoparticle Resuspension Stability

The solid compositions of Examples 3 and 4 were resuspended by adding a 38 mg sample to 2 mL deionized water. The so-formed suspensions were allowed to stand unmixed for 24 hours (ambient conditions) to measure stability. DLS analysis (see Table 4) showed that the average cumulant diameter of the nanoparticles of Example 3 was 96 nm, with a polydispersity of 0.21. The average cumulant diameter of the nanoparticles of Example 4 was 96 nm, with a polydispersity of 0.11. These results demonstrate that a small particle size can be maintained after isolation of the solid composition, and that the resuspended nanoparticles are stable for at least 24 hours with no measurable particle agglomeration.

TABLE 4

| Sample | Condition | Diameter (nm) | Polydispersity |
|---|---|---|---|
| Example 3 | Initial | 85 | 0.10 |
| | After Storage | 86 | 0.10 |
| | After Resuspension | 96 | 0.21 |
| Example 4 | Initial | 85 | 0.10 |
| | After Storage | 85 | 0.10 |
| | After Resuspension | 96 | 0.11 |

### Example 5

For Example 5, nanoparticles containing celecoxib were prepared as follows. First, 120 mg celecoxib and 330 mg ethylcellulose were dissolved in 6 mL methylene chloride to form an organic solution. Next, 150 mg sodium β-caseinate was added to 20 mL deionized water to form an aqueous solution. The organic solution was then poured into the aqueous solution and emulsified as described in Example 1. The methylene chloride was removed from the emulsion using a rotary evaportor, resulting in an aqueous suspension of nanoparticles, with a mass ratio of 20:55:25 celecoxib:ethylcellulose:sodium β-caseinate. DLS analysis showed that the average cumulant diameter of the nanoparticles in suspension was 96 nm, with a polydispersity of 0.19.

### Isolation of Solid Compositions

The nanoparticle suspension of Example 5 was spray-dried as described in Example 1, resulting in the formation of a solid composition of the invention.

### Nanoparticle Resuspension

The solid composition of Example 5 was resuspended by adding a 33.1 mg sample to 3 mL deionized water. DLS analysis showed that the average cumulant diameter of the nanoparticle suspension was 133 nm, with a polydispersity of 0.36.

### Filter Potency

A filter potency test was used to characterize the resuspended nanoparticles of Example 5. A 50 μL sample of the aqueous nanoparticle suspension of Example 5 was added to 1 mL 80/20 methanol/acetonitrile, and the concentration of drug in solution was analyzed by HPLC. The suspension was then filtered using a 0.2 μm filter and diluted in 80/20 methanol/acetonitrile for HPLC analysis. The results of this analysis showed that 87% of the nanoparticle suspension potency is maintained following filtration by a 0.2 μm filter. This indicates that most of the nanoparticles in suspension remained small and unagglomerated.

### Examples 6-10

The nanoparticles of Examples 6-10 were made containing celecoxib, ethylcellulose, and casein, in varying ratios. The amounts of each ingredient used to make Examples 6-10 are shown in Table 5. The nanoparticles were emulsified, and the methylene chloride Was removed, as described in Example 1. Results of DLS analysis of the nanoparticle suspensions are also shown in Table 5.

TABLE 5

| | | Ethyl- | Methylene | | | Cumulant | |
|---|---|---|---|---|---|---|---|
| Sample | Celecoxib | cellulose | chloride | Casein | Water | Diameter | |
| (mass ratio)* | (mg) | (mg) | (mL) | (mg) | (mL) | (nm) | Polydispersity |
| Example 6 | 75 | 225 | 5 | 100 | 20 | 121 | 0.29 |
| (19:56:25) | | | | | | | |
| Example 7 | 1800 | 1800 | 80 | 1200 | 240 | 144 | 0.26 |
| (37.5:37.5:25) | | | | | | | |
| Example 8 | 187.5 | 112.5 | 5 | 100 | 20 | 118 | 0.20 |
| (47:28:25) | | | | | | | |
| Example 9 | 210 | 90 | 5 | 100 | 20 | 126 | 0.25 |
| (52.5:22.5:25) | | | | | | | |
| Example 10 | 225 | 75 | 5 | 100 | 20 | 122 | 0.27 |
| (56:19:25) | | | | | | | |

*mass ratio of celecoxib:ethylecellulose:casein

The aqueous suspensions of Examples 6-10 were allowed to stand unmixed at 5° C. to measure stability. The results of DLS analysis are shown in Table 6. These results demonstrate that the nanoparticle suspensions are stable during storage with no significant particle agglomeration.

TABLE 6

| | Storage | Initial | | After Storage | |
|---|---|---|---|---|---|
| | Du- | Di- | | Di- | |
| | ration | ameter | | ameter | |
| Sample | (days) | (nm) | Polydispersity | (nm) | Polydispersity |
| Example 6 | 1 | 121 | 0.29 | 121 | 0.25 |
| Example 7 | 8 | 144 | 0.26 | 148 | 0.27 |
| Example 8 | 1 | 118 | 0.20 | 119 | 0.20 |
| Example 9 | 3 | 126 | 0.25 | 122 | 0.16 |
| Example | 3 | 122 | 0.27 | 124 | 0.23 |
| 10 | | | | | |

### Isolation of Solid Compositions

The nanoparticles of Examples 6-10 were spray-dried as described in Example 1, resulting in the formation of solid compositions of the invention.

### Nanoparticle Resuspension

The solid compositions of Examples 6-10 were resuspended by adding a sufficient amount of sample to obtain 20 mg/mL solids in deionized water. The results of DLS analysis are shown in Table 7. This demonstrates that the small particle size was maintained after isolation of the nanoparticles in dry powder form, followed by resuspension.

TABLE 7

| | Initial | | After Resuspension | |
|---|---|---|---|---|
| | Diameter | | Diameter | |
| Sample | (nm) | Polydispersity | (nm) | Polydispersity |
| Example 6 | 121 | 0.29 | 125 | 0.26 |
| Example 7 | 144 | 0.26 | 150 | 0.27 |
| Example 8 | 118 | 0.20 | 131 | 0.28 |
| Example 9 | 126 | 0.25 | 116 | 0.27 |
| Example 10 | 122 | 0.27 | 121 | 0.32 |

### Filter Potency

A filter potency test was used to characterize the resuspended nanoparticles of Examples 6-10. First, a 25 μL sample of the aqueous nanoparticle suspension was added to 975 μL 80/20 acetonitrile/methanol, and the concentration of drug in solution was analyzed by HPLC. Next, the suspension was filtered using a 0.45 μm filter and diluted in 80/20 methanol/acetonitrile for HPLC analysis.

Potencies of the nanoparticle suspensions are shown in Table 8. The results in TO Table 8 show that all of the samples retained high potency, indicating that the nanoparticles in suspension remained small and unagglomerated.

TABLE 8

| | | Potency | |
|---|---|---|---|
| | Potency | 0.45 μm | |
| | Unfiltered | filtered | Potency |
| Sample | (mg/mL) | (mg/mL) | Retained (%) |
| Example 6 | 3.10 | 3.05 | 98 |
| Example 7 | 7.10 | 6.71 | 95 |
| Example 8 | 4.85 | 4.55 | 94 |
| Example 9 | 4.17 | 4.09 | 98 |
| Example 10 | 3.72 | 3.53 | 95 |

### Example 11

For Example 11, nanoparticles containing celecoxib were prepared as follows. First, 96 mg celecoxib and 336 mg ethylcellulose were dissolved in 6 mL methylene chloride to form an organic solution. Next, 48 mg sodium taurocholate (NaTC) as a surface stabilizer was added to 24 mL deionized water to form an aqueous solution. The organic solution was then poured into the aqueous solution and emulsified as described above. The methylene chloride was removed from the emulsion using a rotary evaporator, resulting in an aqueous suspension of nanoparticles, with a composition ratio of 20:70:10 celecoxib:ethylcellulose:NaTC. DLS analysis showed that the average cumulant diameter of the nanoparticles in suspension was 73 nm, with a polydispersity of 0.17. A nanoparticle suspension of the present invention was formed by adding 120 mg casein (5 mg/mL) to the aqueous nanoparticle suspension, resulting in a mass ratio of 16:56:8:20 celecoxib:ethylcellulose:NaTC:casein.

### Isolation of Solid Compositions

The nanoparticle suspension of the present invention was spray dried as described in Example 1, resulting in the formation of a solid composition of the present invention.

### Nanoparticle Resuspension

The solid composition of Example 11 was resuspended by adding a 25 mg sample to 1.1 mL deionized water. DLS analysis showed that the average cumulant diameter of the nanoparticle suspension was 107 nm, with a polydispersity of 0.20. This demonstrates that a small particle size can be obtained after isolation of the solid composition, followed by resuspension.

### Filter Potency

A filter potency test was used to characterize the resuspended nanoparticles of Example 11. A 100 μL sample of the aqueous nanoparticle suspension of Example 11 was added to 1 mL 80/20 methanol/acetonitrile, and the concentration of drug in solution was analyzed by HPLC. Next, the suspension was filtered using a 0.2 μm filter and diluted in 80/20 methanol/acetonitrile for HPLC analysis.

Potencies of the nanoparticle suspensions are shown in Table 9. The results in Table 9 show that 94% of the nanoparticle suspension potency is maintained following filtration by a 0.2 μm filter. This indicates that most of the nanoparticles in suspension remain small and unagglomerated.

TABLE 9

| | | Potency | |
|---|---|---|---|
| | Potency | 0.2 μm | |
| | Unfiltered | filtered | Potency |
| Sample | (mg/mL) | (mg/mL) | Retained (%) |
| Example 11 | 3.4 | 3.2 | 94 |

### Examples 12 and 13

The nanoparticles of Examples 12 and 13 were made containing celecoxib, ethylcellulose, and NaTC, and spray-dried with two concentrations of casein. For the nanoparticles of Example 12, 240 mg celecoxib and 300 mg ethylcellulose were dissolved in 7.5 mL methylene chloride to form an organic solution, and 60 mg NaTC was added to 30 mL deionized water to form an aqueous solution. For the nanoparticles of Example 13, 8 g celecoxib and 10 g ethylcellulose were dissolved in 300 mL methylene chloride to form an organic solution, and 2 g NaTC was added to 1 L deionized water to form an aqueous solution. The solutions were mixed and emulsified as described for Example 1. The methylene chloride was removed using a rotary evaporator, to obtain the aqueous suspensions of nanoparticles of Examples 12 and 13. The nanoparticles of Examples 12 and 13 both had mass ratios of 40:50:10 celecoxib:ethylcellulose:NaTC. DLS analysis showed that the average cumulant diameter of the nanoparticles of Example 12 was 69 nm, with a polydispersity of 0.19. The average cumulant diameter of the nanoparticles of Example 13 was 90 nm, with a polydispersity of 0.08.

Nanoparticle suspensions of the present invention were formed by adding 150 mg casein (5 mg/mL) to the aqueous nanoparticle suspension of Example 12, and 6.67 mg casein to the aqueous nanoparticle suspension of Example 13. The nanoparticle suspension of Example 12 had a mass ratio of 32:40:8:20 celecoxib:ethylcellulose:NaTC:casein, while the nanoparticle suspension of Example 13 had a mass ratio of 30:37.5:7.5:25 celecoxib:ethylcellulose:NaTC:casein.

### Isolation of Solid Compositions

Solid compositions of the invention were prepared by spray drying the nanoparticle suspension of Examples 12 using the procedure described in Example 1. The nanoparticle suspension of Example 13 was spray dried as follows. The nanoparticle suspension was pumped to a Niro type XP Portable Spray-Drier with a Liquid-Feed Process Vessel (“PSD-1”), equipped with a pressure nozzle (Schlick 1.0; Dusen Schlick, GmbH of Untersiemau, Germany). The PSD-1 was equipped with 9-inch and 4-inch chamber extensions. The chamber extensions were added to the spray drier to increase the vertical length of the dryer. The added length increased the residence time within the drier, which allowed the product to dry before reaching the angled section of the spray dryer. The nanoparticle suspension was pumped to the spray drier at about 20 g/min at a pressure of 175 psig. Drying gas (nitrogen) was introduced into the chamber at an inlet temperature of 90° C. The evaporated solvent and drying gas exited the spray drier at a temperature of 50° C. The resulting solid composition was collected in a cyclone.

### Nanoparticle Resuspension

The solid composition of Example 12 was resuspended by adding a 37 mg sample to 2 mL deionized water. DLS analysis showed that the average cumulant diameter of the nanoparticles of Example 12 was 88 nm, with a polydispersity of 0.19.

The solid composition of Example 13 was stored in a sealed container at room temperature for 66 days to evaluate storage stability of the nanoparticles in dried form. The effect of storage on nanoparticle agglomeration was determined by resuspending the aged sample and analyzing the particle size in the suspension. The aged solid compositions of Example 13 were resuspended by adding a 25 mg sample to 1 mL deionized water. The average cumulant diameter of the nanoparticles of Example 13 was 110 nm, with a polydispersity of 0.02. This demonstrates successful resuspension of the nanoparticles, maintaining small particle size, and storage of solid compositions without particle agglomeration.

### Controls 2-6

For Controls 2-6, nanoparticles were made as described above containing celecoxib, ethylcellulose, and NaTC, in the same ratio as for Examples 12 and 13 (40:50:10), except that they were spray-dried without casein. Instead of casein, other matrix materials were added to the nanoparticle suspension prior to spray drying. Table 10 shows the compositions of Controls 2-6.

TABLE 10

| | | Nanoparticle Composition (mass ratio |
|---|---|---|
| Sample | Matrix Material | celecoxib:ethylcellulose:NaTC:matrix) |
| Control 2 | acacia | 32:40:8:20 |
| Control 3 | acacia | 30:37.5:7.5:25 |
| Control 4 | trehalose | 20:25:5:50 |
| Control 5 | lactose | 20:25:5:50 |
| Control 6 | mannitol | 20:25:5:50 |

### Nanoparticle Resuspension

The solid compositions of Controls 2 and 3 were resuspended by adding about 25 mg/mL solids to deionized water. The results of DLS analysis are shown in Table 11. DLS results from resuspension of the solid compositions of Examples 12 and 13 are shown for comparison. The data in Table 11 show that, for nanoparticles of the invention, a small particle size can be maintained after drying and resuspension. However, for the control nanoparticles made without casein, the average diameter of resuspended particles is outside the invention.

TABLE 11

| | Before Formation of | | | |
|---|---|---|---|---|
| | Solid Composition | | After Resuspension | |
| | Diameter | | Diameter | |
| Sample | (nm) | Polydispersity | (nm) | Polydispersity |
| Example 12 | 69 | 0.19 | 88 | 0.19 |
| Example 13 | 90 | 0.08 | 110 | 0.02 |
| Control 2 | 69 | 0.19 | 547 | 0.74 |
| Control 3 | 90 | 0.08 | 543 | 0.65 |

### Filter Potency

A filter potency test was used to characterize the resuspended nanoparticles of Examples 12 and 13, and Controls 2-6. First, a 25 μL sample of the aqueous nanoparticle suspension was added to 975 μL 80/20 acetonitrile/methanol, and the concentration of drug in solution was analyzed by HPLC. Next, the suspension was filtered through a 0.2 μm filter and diluted in 80/20 methanol/acetonitrile for HPLC analysis.

Potencies of the nanoparticle suspensions are shown in Table 12. The results in Table 12 show that greater than 94% of the nanoparticle suspension potency is maintained following filtration of Examples 12 and 13. However, 29% or less of the nanoparticle suspension potency is maintained following filtration of, Controls 2-6. This indicates the nanoparticles of the invention remain small and unagglomerated, while the nanoparticles of the controls agglomerate in solution.

TABLE 12

| | | Potency | |
|---|---|---|---|
| | Potency | 0.2 μm | |
| | Unfiltered | filtered | Potency |
| Sample | (mg/mL) | (mg/mL) | Retained (%) |
| Example 12 | 7.13 | 6.96 | 98 |
| Example 13 | 7.01 | 6.61 | 94 |
| Control 2 | 7.77 | 0.06 | <1 |
| Control 3 | 6.02 | 0.05 | <1 |
| Control 4 | 5.46 | 1.57 | 29 |
| Control 5 | 5.07 | 1.16 | 23 |
| Control 6 | 5.31 | 0.33 | 6 |

### Example 14

For Example 14, nanoparticles containing celecoxib were prepared as follows. First, 120 mg celecoxib and 420 mg ethylcellulose were dissolved in 7.5 mL methylene chloride to form an organic solution. Next, 60 mg sodium taurocholate (NaTC) was added to 30 mL deionized water to form an aqueous solution. The organic solution was then poured into the aqueous solution and emulsified as described in Example 1. The methylene chloride was removed from the emulsion using a rotary evaporator, resulting in an aqueous suspension of nanoparticles, with a mass ratio of 20:70:10 celecoxib:ethylcellulose:NaTC. DLS analysis showed that the average cumulant diameter of the nanoparticles in suspension was 63 nm, with a polydispersity of 0.08. A nanoparticle suspension of the invention was obtained by adding 166.3 mg sodium caseinate to this aqueous suspension. The nanoparticle suspension of Example 14 had a mass ratio of 15:52.5:7.5:25 celecoxib:ethylcellulose:NaTC:casein.

### Isolation of Solid Compositions

A solid composition of the invention was obtained by spray drying the nanoparticle suspension of Example 14 as described in Example 1.

### Nanoparticle Resuspension

The solid composition of Example 14 was resuspended by adding a 37.7 mg sample to 4 mL deionized water. DLS analysis showed that the average cumulant diameter of the nanoparticle suspension was 107 nm, with a polydispersity of 0.34. This demonstrates that a small particle size can be maintained after isolation of the solid composition, followed by resuspension.

### Controls 7 and 8

For Control 7 and 8, nanoparticles were made as described in Example 14 containing celecoxib, ethylcellulose, and NaTC, in the same ratio as for Example 14 (20:70:10), except that they were spray-dried with different amounts of acacia as the matrix material to form a solid composition. Table 13 shows the compositions of Controls 7 and 8.

TABLE 13

| | | Nanoparticle Composition (mass ratio |
|---|---|---|
| Sample | Matrix Material | celecoxib:ethylcellulose:NaTC:matrix) |
| Control 7 | acacia | 30:37:5:7.5:25 |
| Control 8 | acacia | 20:25:5:50 |

### Nanoparticle Resuspension and Filter Potency Tests

The solid compositions of Controls 7 and 8 were resuspended by adding 27 mg/mL of Control 7 nanoparticles, or 40 mg/mL of Control 8 nanoparticles to deionized water. Example 14 nanoparticles were resuspended as described above. A filter potency test was used to characterize the resuspended nanoparticles of Example 14, and Controls 7 and 8. First, a 25 μL sample of the aqueous nanoparticle suspension was added to 975 μL 80/20 acetonitrile/methanol, and the concentration of drug in solution was analyzed by HPLC. Next, the suspension was filtered using a 0.2 μm filter and diluted in 80/20 methanol/acetonitrile for HPLC analysis.

Potencies of the nanoparticle suspensions are shown in Table 14. The results in Table 14 show that 90% of the nanoparticle suspension potency is maintained following filtration of Example 14. However, 13% or less of the nanoparticle suspension potency is maintained following filtration of Controls 7 and 8. This indicates the nanoparticles of Example 14 remained small and unagglomerated, while the nanoparticles of the controls agglomerate in solution.

TABLE 14

| | | Potency | |
|---|---|---|---|
| | Potency | 0.2 μm | |
| | Unfiltered | filtered | Potency |
| Sample | (mg/mL) | (mg/mL) | Retained (%) |
| Example 14 | 1.19 | 1.07 | 90 |
| Control 7 | 2.09 | 0.27 | 13 |
| Control 8 | 1.17 | 0.02 | 2 |

### Control 9

The nanoparticles of Control 9 were made containing celecoxib and casein, without the non-ionizable polymer, using the procedures described in Example 1 with the following exceptions. The organic solution consisted of 150.0 mg celecoxib dissolved in 6 mL methylene chloride, while the aqueous solution consisted of 454.8 mg sodium caseinate (Spectrum Chemicals) in 20 mL deionized water. The methylene chloride was removed from the emulsion using a rotary evaporator, resulting in an aqueous suspension of nanoparticles. DLS analysis showed that the average cumulant diameter of the nanoparticles in suspension was 97 nm, with a polydispersity of 0.31. However, crystals were visible in the suspension within 10 minutes, and the suspension appeared cloudier over time.

### Filter Potency

A filter potency test was used to characterize the nanoparticle suspension of Control 9. A 100 μL sample of the aqueous nanoparticle suspension was added to 1 mL 80/20 acetonitrile/methanol, and the concentration of drug in solution was analyzed by HPLC. Next, the suspension was filtered using a 1 μm glass membrane filter and diluted in 80/20 methanol/acetonitrile for HPLC analysis.

Potencies of the nanoparticle suspensions are shown in Table 15. The results in Table 15 show that 96% of the nanoparticle suspension potency was lost following filtration by a 1 μm filter. This indicates that without the non-ionizable polymer, the nanoparticles are not stable in suspension.

TABLE 15

| | Potency | Potency | |
|---|---|---|---|
| | Unfiltered | 1 μm filtered | Potency |
| Sample | (mg/mL) | (mg/mL) | Retained (%) |
| Control 9 | 5.74 | 0.23 | 4 |

### Examples 15 and 16

Nanoparticles containing Drug 2 were made using the procedures outlined in Example 1 with the following exceptions. For Example 15, the organic solution contained 300 mg Drug 2 and 300 mg ethylcellulose in 7.5 mL ethyl acetate. This organic solution was mixed with 30 mL deionized water and emulsified using the procedures outlined in Example 1 to form nanoparticles having a cumulant diameter of 178 nm and a polydispersity of 0.12. For Example 16, the organic solution contained 300 mg Drug 2 and 300 mg pCL-PEG in 7.5 mL ethyl acetate. This organic solution was mixed with 30 mL deionized water and emulsified using the procedures outlined in Example 1 to form nanoparticles having a cumulant diameter of 117 nm and a polydispersity of 0.19. Results of DLS analysis of these nanoparticle suspensions are shown in Table 16.

TABLE 16

| | | Cumulant | |
|---|---|---|---|
| | | Diameter | |
| Sample | Composition | (nm) | Polydispersity |
| Example 15 | 50:50 Drug 2:Ethylcellulose | 178 | 0.12 |
| Example 16 | 50:50 Drug 2:pCL-PEG | 117 | 0.19 |

Nanoparticles suspensions of the present invention were then formed by adding casein to the above nanoparticle suspensions. For Example 15, the resulting nanoparticle suspension consisted of 40:40:20 Drug 2:ethylcellulose:casein. For Example 16, the resulting nanoparticle suspension consisted of 40:40:20 Drug 2:pCL-PEG:Casein.

### Isolation of Solid Compositions

The nanoparticle suspensions of Examples 15 and 16 were spray-dried as described in Example 1 to form solid compositions of the invention.

### Nanoparticle Resuspension and Filter Potency

The solid compositions of Examples 15 and 16 were resuspended by adding a 38 mg sample to 2 mL deionized water. A filter potency test was used to characterize the resuspended nanoparticles of Examples 15 and 16. First, a 100 μL sample of the aqueous nanoparticle suspension was added to 0.5 mL methanol, and the concentration of drug in solution was analyzed by HPLC. Next, the suspension was filtered using a 1 μm filter and diluted in methanol for HPLC analysis.

Potencies of the nanoparticle suspensions are shown in Table 17. The results in Table 17 show that 99% of the nanoparticle suspension potency is maintained following filtration of Example 15, and 73% of the nanoparticle suspension potency is maintained following filtration of Example 16.

TABLE 17

| | Potency | Potency | |
|---|---|---|---|
| | Unfiltered | 1 μm filtered | Potency |
| Sample | (mg/mL) | (mg/mL) | Retained (%) |
| Example 15 | 6.043 | 5.998 | 99 |
| Example 16 | 8.705 | 6.324 | 73 |

### Example 17

Nanoparticles containing Drug 3 were prepared as follows. First, 120 mg Drug 3 and 420 mg ethylcellulose were dissolved in 7.5 mL methylene chloride to form an organic solution. Next, 60 mg NaTC (a surface stabilizer) was added to 30 mL deionized water to form an aqueous solution. The organic solution was then poured into the aqueous solution and emulsified as described in Example 1. The methylene chloride was removed from the emulsion using a rotary evaporator, resulting in an aqueous suspension of nanoparticles, with a composition ratio of 20:70:10 Drug 3:ethylcellulose:NaTC. DLS analysis showed that the average cumulant diameter of the nanoparticle suspension was 64 nm, with a polydispersity of 0:20.

A nanoparticle suspension of the present invention was formed by adding casein to this suspension, resulting in nanoparticle suspension consisting of 16:56:8:20 Drug 3:ethylcellulose:NaTC:casein.

### Isolation of Solid Compositions

The nanoparticle suspension of Example 17 was spray-dried as described in Example 1, resulting in the formation of a solid composition of the invention.

### Nanoparticle Resuspension

The solid composition of Example 17 was resuspended by adding 25 mg of sample to 1 mL deionized water. DLS analysis showed that the average cumulant diameter of the nanoparticle suspension was 143 nm, with a polydispersity of 0.30. This demonstrates that a small particle size can be maintained after isolation of the nanoparticles in dry powder form, followed by resuspension.

### Filter Potency

A filter potency test was used to characterize the resuspended nanoparticles of Example 17. First, a 50 μL sample of the aqueous nanoparticle suspension was added to 1 mL methanol, and the concentration of drug in solution was analyzed by HPLC. Next, the suspension was filtered using a 0.2 μm filter and diluted in methanol for HPLC analysis.

Potencies of the nanoparticle suspensions are shown in Table 18. The results in Table 18 show that 98% of the nanoparticle suspension potency is maintained following filtration of the resuspended nanoparticles of Example 17 using a 0.2 μm filter. This indicates that most of the nanoparticles in suspension remain small and unagglomerated.

TABLE 18

| | | Potency | |
|---|---|---|---|
| | Potency | 0.2 μm | |
| | Unfiltered | filtered | Potency |
| Sample | (mg/mL) | (mg/mL) | Retained (%) |
| Example 17 | 4.3 | 4.2 | 98 |

### Examples 18 and 19

The nanoparticles of Examples 18 and 19 were made containing Drug 4, ethylcellulose, and sodium caseinate (Spectrum Chemicals). The materials and amounts used to make the nanoparticle suspensions of Examples 18 and 19 are shown in Table 19. The nanoparticles were emulsified, and the methylene chloride was removed, as described in Example 1.

TABLE 19

| Sample | Drug | | Methylene | | |
|---|---|---|---|---|---|
| (composition | 4 | Ethylcellulose | Chloride | Casein | Water |
| ratio)* | (mg) | (mg) | (mL) | (mg) | (mL) |
| Example 18 | 1500 | 1500 | 40 | 1000 | 200 |
| (37.5:37.5:25) | | | | | |
| Example 19 | 750 | 2250 | 40 | 1000 | 200 |
| (18.75:56.25:25) | | | | | |

*Composition Ratio: mass ratio of Drug 4: ethylcellulose:casein

### Isolation of Solid Compositions

The nanoparticle suspensions of Examples 18 and 19 were spray-dried using the PSD-1 spray-drier, as described for Example 13, forming solid compositions of the invention.

### Nanoparticle Resuspension

The solid compositions were resuspended by adding 30 mg of sample to 1.5 mL deionized water (Example 18), or 40 mg of sample to 2 mL deionized water (Example 19). Samples were vortexed 30 seconds to suspend the nanoparticles. DLS analysis showed that the average cumulant diameter of the nanoparticle resuspension of Example 18 was 121 nm, with a polydispersity of 0.21. The average cumulant diameter of the nanoparticle resuspension of Example 19 was 138 nm, with a polydispersity of 0.17. This demonstrates that a small particle size can be maintained after isolation of the solid composition, followed by resuspension.

### Filter Potency

A filter potency test was used to characterize the resuspended nanoparticles of Examples 18 and 19. First, a 25 μL sample of the aqueous nanoparticle suspension was added to 975 μL methanol, and the concentration of drug in solution was analyzed by HPLC. Next, the suspension was filtered using a 0.45 μm filter and diluted in methanol for HPLC analysis.

Potencies of the nanoparticle suspensions are shown in Table 20. The results in Table 20 show that greater than 85% of the nanoparticle suspension potency is maintained following filtration of Examples 18 and 19 using a 0.45 μm filter. This indicates that the nanoparticles in suspension remain small and unagglomerated.

TABLE 20

| | | Potency | |
|---|---|---|---|
| | Potency | 0.45 μm | |
| | Unfiltered | filtered | Potency |
| Sample | (mg/mL) | (mg/mL) | Retained (%) |
| Example 18 | 5.52 | 5.37 | 97 |
| Example 19 | 3.92 | 3.73 | 95 |

### Control 10

The nanoparticles of Control 10 were made containing Drug 4 and casein, without the non-ionizable polymer using the procedures described in Example 1 with the following exceptions. The organic solution consisted of 149.6 mg Drug 4 dissolved in 6 mL methylene chloride, while the aqueous solution consisted of 451.7 mg sodium caseinate in 20 mL deionized water. The methylene chloride was removed from the emulsion using a rotary evaporator, resulting in an aqueous suspension of nanoparticles. DLS analysis showed that the average cumulant diameter of the nanoparticles in suspension was 288 nm, with a polydispersity of 0.25. The suspension appeared opaque.

### PXRD Evaluation

The nanoparticles of Control 10 were examined using powder x-ray diffraction (PXRD) with a Bruker AXS D8 Advance diffractometer to determine the amorphous or crystalline character of the drug in the nanoparticles. Samples (approximately 100 mg) were packed in Lucite sample cups fitted with Si(511) plates as the bottom of the cup to give no background signal. Samples were spun in the cp plane at a rate of 30 rpm to minimize crystal orientation effects. The x-ray source (KCu_{α}, λ=1.54 Å) was operated at a voltage of 45 kV and a current of 40 mA. Data for each sample were collected over a period of 27 minutes in continuous detector scan mode at a scan speed of 1.8 seconds/step and a step size of 0.04°/step. Diffractograms were collected over the 20 range of 4° to 40° and exhibited sharp peaks characteristic of crystalline drug. These data indicate that a significant portion of the drug in the nanoparticles of Control 10 was in crystalline form. Thus, including a non-ionizable polymer in the nanoparticles of the present invention helps maintain the drug in a non-crystalline form.

### Example 20

Nanoparticles containing triphenyl bismuth (Drug 5), were prepared as follows. First, 40 mg Drug 5 and 280 mg ethylcellulose were dissolved in 5 mL methylene chloride to form an organic solution. Next, 80 mg NaTC (a surface stabilizer) was added to 20 mL deionized water to form an aqueous solution. The organic solution was then poured into the aqueous solution and emulsified as described in Example 1. The methylene chloride was removed from the emulsion using a rotary evaporator, resulting in an aqueous suspension of nanoparticles, with a composition ratio of 10:70:20 Drug 5:ethylcellulose:NaTC.

Nanoparticle suspensions of the invention were formed by adding 67 mg sodium caseinate to 10 mL of the above suspension, resulting in a nanoparticle suspension with a mass ratio of 7.5:52.5:15:25 Drug 5:ethylcellulose:NaTC:casein.

### Isolation of Solid Compositions

The nanoparticle suspension of Example 20 was spray-dried using the procedures described in Example 1.

### Control 11

For Control 11, the 10:70:20 Drug 5:ethylcellulose:NaTC nanoparticles were spray-dried using the procedures described in Example 1. No sodium caseinate was added to the suspensions prior to spray drying.

### Nanoparticle Resuspension

The solid compositions of Example 20 were resuspended by adding 53 mg of sample to 2 mL deionized water, and vortexing for 30 seconds. The solid composition of Control 11 was resuspended by adding 40 mg of sample to 2 mL deionized water, and vortexing for 30 seconds. DLS analysis showed that the average cumulant diameter of the nanoparticle suspension of Example 20 was 146 nm, with a polydispersity of 0.49. The average cumulant diameter of the nanoparticle suspension of Control 11 was 1258 nm, with a polydispersity of 0.61. This demonstrates that including casein in the formulation is necessary to maintain a small particle size following resuspension.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

1. A solid pharmaceutical composition comprising:
(a) nanoparticles comprising a poorly water soluble drug and a poorly aqueous soluble non-ionizable polymer, wherein
(i) said poorly water soluble drug has a solubility in water of less than 5 mg/mL over the pH range of 6.5 to 7.5;
(ii) at least 90 wt % of said drug in said nanoparticles is in a non-crystalline form;
(iii) said nanoparticles have an average size of less than 500 nm; and
(iv) the mass ratio of said poorly water soluble drug to said poorly aqueous soluble non-ionizable polymer is less than 9:1; and
(b) casein or a pharmaceutically acceptable form thereof
wherein the mass ratio of (1) said casein to (2) the combined mass of said poorly water soluble drug and said poorly aqueous soluble non-ionizable polymer is at least 1:20.

2. The composition of claim 1 wherein said mass ratio of (1) said casein to (2) the combined mass of said poorly water soluble drug and said poorly aqueous soluble non-ionizable polymer is at least 1:10.

3. The composition of claim 1 wherein said poorly water soluble drug, said poorly aqueous soluble non-ionizable polymer, and said casein constitute at least 80 wt % of said composition.

4-6. (canceled)

7. The composition of claim 1 wherein said mass ratio of said poorly water soluble drug to said poorly aqueous soluble non-ionizable polymer ranges from 1:19 to 3:1.

8. The composition of claim 1 wherein said poorly aqueous soluble non-ionizable polymer is selected from the group consisting of ethylcellulose, propylcellulose, butylcellulose, cellulose acetate, cellulose propionate, cellulose butyrate, cellulose acetate propionate, cellulose acetate butyrate, methyl cellulose acetate, methyl cellulose propionate, methyl cellulose butyrate, ethyl cellulose acetate, ethyl cellulose propionate, ethyl cellulose butyrate, hydroxypropyl methylcellulose acetate, hydroxypropyl methylcellulose propionate, hydroxypropyl methylcellulose butyrate, poly(vinyl acetate), poly(vinyl acetate-co-vinyl alcohol), poly(ethylene-co-vinyl acetate), poly(ethyl acrylate-methyl methacrylate) (2:1 monomer ratio), poly(lactide), poly(glycolide), poly(ε-caprolactone), poly(lactide-co-glycolide), poly(lactide-co-ε-caprolactone), poly(ethylene oxide-co-ε-caprolactone), poly(ethylene oxide-co-lactide), poly(ethylene oxide-co-lactide-co-glycolide), poly(isobutyl)cyanoacrylate, and poly(hexyl)cyanoacrylate.

9. The composition of claim 1 wherein said poorly aqueous soluble non-ionizable polymer is selected from the group consisting of ethylcellulose and poly(ethylene oxide-co-ε-caprolactone).

10. The composition of claim 1 wherein said casein is selected from the group consisting of αₛ₁-casein, αₛ₂-casein, β-casein, κ-casein, vegetable casein, sodium caseinate, calcium caseinate, potassium caseinate, ammonium caseinate, and mixtures thereof.

11. The composition of wherein said nanoparticles further comprise a surface stabilizer.

12. The composition of claim 9 wherein said poorly water soluble drug, said poorly aqueous soluble non-ionizable polymer, said surface stabilizer, and said casein constitute at least 90 wt % of said composition.

13. The composition of claim 9 wherein said composition consists essentially of said poorly water soluble drug, said poorly aqueous soluble non-ionizable polymer, said surface stabilizer, and said casein.

14. The composition of claim 9 wherein said surface stabilizer is selected from the group consisting of casein, caseinates, polyvinyl pyrrolidone, polyoxyethylene alkyl ethers, polyoxyethylene stearates, polyoxyethylene castor oil derivatives, poly(ethylene oxide-propylene oxide), tragacanth, gelatin, polyethylene glycol, bile salts, phospholipids, sodium dodecylsulfate, benzalkonium chloride, sorbitan esters, polyoxyethylene alkyl ethers, polyoxyethylene castor oil derivatives, polyoxyethylene sorbitan fatty acid esters, polyoxyethylene stearates, triethanolamine, sodium docusate, sodium stearyl fumarate, sodium cyclamate, and mixtures and pharmaceutically acceptable forms thereof.

15. The composition of claim 1 wherein said composition comprises 1 wt % to 60 wt % said poorly aqueous soluble drug, 10 wt % to 80 wt % said poorly aqueous soluble non-ionizable polymer, and 10 wt % to 50 wt % said casein.

16. The composition of claim 1 wherein said poorly water soluble drug and said poorly aqueous soluble non-ionizable polymer are present in said nanoparticle in the form of a solid solution.

17. The composition of wherein said nanoparticles are encapsulated within said casein.

18. The composition of wherein said nanoparticles comprise said casein.

19. A pharmaceutical composition comprising an aqueous suspension, said aqueous suspension comprising:
(a) nanoparticles comprising a poorly water soluble drug and a poorly aqueous soluble non-ionizable polymer, wherein
(i) said poorly water soluble drug has a solubility in water of less than 5 mg/mL over the pH range of 6.5 to 7.5,
(ii) at least 90 wt % of said drug in said nanoparticles is in a non-crystalline form,
(iii) said nanoparticles have an average size of less than 500 nm;
(iv) said poorly water soluble drug and said poorly aqueous soluble non-ionizable polymer constitute at least 60 wt % of said nanoparticles, and
(v) the mass ratio of said poorly water soluble drug to said poorly aqueous soluble non-ionizable polymer is less than 9:1;
(b) casein or a pharmaceutically acceptable form thereof; and
(c) water.

20. A process for forming nanoparticles, comprising:
(a) forming an organic solution comprising a poorly water soluble drug and a poorly aqueous soluble non-ionizable polymer dissolved in an organic solvent, wherein
(i) said drug has a solubility in water of less than 5 mg/ml over the pH range of 6.5 to 7.5, and
(ii) the mass ratio of said poorly water soluble drug to said poorly aqueous soluble non-ionizable polymer is less than 9:1;
(b) forming an aqueous solution;
(c) mixing said organic solution with said aqueous solution to form a first mixture;
(d) removing said organic solvent from said first mixture to form a suspension comprising said nanoparticles and said aqueous solution, wherein
(i) said nanoparticles have an average size of less than 500 nm, and
(ii) at least 90 wt % of said drug in said nanoparticles is non-crystalline; and
(e) adding casein or a pharmaceutically form thereof to either said aqueous solution of step (b) or to said suspension of step (d), wherein the mass ratio of (1) said casein to (2) the combined mass of said poorly water soluble drug and said poorly aqueous soluble non-ionizable polymer is at least 1:20.
